(19) United States
(12) Patent Application Publication
(10) Pub. No.: US 2026/0076642 A1
(43) Pub. Date: Mar. 19, 2026

(54) ACOUSTIC MEASURING DEVICE FOR MONITORING OF HEALTH DATA

(71) Applicant: Esperto Medical, Inc., Irvine, CA (US)

(72) Inventors: Aditya Rajagopal, Irvine, CA (US); Alaina Brinley Rajagopal, Irvine, CA (US); Vernon Norviel, Evergreen, CO (US)

(21) Appl. No.: 19/399,194

(22) Filed: Nov. 24, 2025

Related U.S. Application Data
(63) Continuation-in-part of application No. 18/589,357, filed on Feb. 27, 2024.
(60) Provisional application No. 63/448,622, filed on Feb. 27, 2023.
(60) Provisional application No. 63/527,543, filed on Jul. 18, 2023.
(60) Provisional application No. 63/527,549, filed on Jul. 18, 2023.

(51) Int. Cl. A61B 8/04; A61B 8/00; G16H 50/30

(52) U.S. Cl. CPC A61B 8/04; A61B 8/4209; G16H 50/30

(57) ABSTRACT

The present disclosure provides devices and methods for monitoring health data of a subject. A device may include a wearable acoustic unit and a processing unit. The wearable acoustic unit may non-invasively detect an acoustic signal from a blood vessel of a subject. The processing unit may use the acoustic signal to continuously health data of a subject. Monitoring health data may include continuously monitoring a subject's blood pressure at an error rate of less than or equal to two percent.

### CROSS-REFERENCE TO RELATED APPLICATION

This application is a continuation-in-part of U.S. patent application Ser. No. 18/589,357, filed Feb. 27, 2024, which claims the benefit of U.S. Provisional Patent Application No. 63/448,622, filed on Feb. 27, 2023, U.S. Provisional Patent Application No. 63/527,543, filed on Jul. 18, 2023, and U.S. Provisional Patent Application No. 63/527,549, filed on Jul. 18, 2023, each of which applications is entirely incorporated herein by reference.

### BACKGROUND

Blood pressure is an essential vital sign routinely used to manage patient care. Methods of blood pressure measurement are typically cumbersome and bulky. Typical methods, such as using stethoscopes in conjunction with a sphygmomanometer and blood pressure arm/wrist cuffs, have several limitations, including susceptibility to ambient noise, patient discomfort, and inability to obtain a continuous blood pressure measurement. As an alternative to the blood pressure cuff, providers may implement invasive blood pressure measurement techniques, such as inserting an arterial catheter. While arterial catheters can provide higher accuracy and data quality than an external cuff, the invasive nature of this technique produces much higher risks, including infection, hemorrhage, or ischemia. Alternative non-invasive modalities for measuring blood pressure are highly desirable, particularly as hypertension has become an increasingly prevalent medical issue in both the United States and the rest of the world.

Furthermore, since existing blood pressure measurement devices are not capable of accurate, non-invasive, and continuous measurements, it is not possible and/or practical, to monitor for real-time changes in blood pressure outside of a hospital setting. This impedes the ability of existing blood pressure measurement devices to monitor blood pressure changes in real-time to determine and/or monitor for changes in individual health conditions and/or pathologies, including, e.g., monitoring for conditions that cause and/or are attributable to acute hypertension and/or hypotension.

### SUMMARY

Owing to the complementary diagnostic information provided by stethoscopes and audio systems, there is a need for systems and methods that utilize both of these technologies. Ideally, such systems and methods would also measure and incorporate information regarding physiological parameters, such as heart rate, blood pressure, body temperature, respiration rate, or SpO2 (saturation of hemoglobin with O2).

The systems and methods described herein are generally directed to a non-invasive blood pressure measuring device providing enhanced functionality over other blood pressure measuring devices that are commonly used by medical professionals. An enhanced non-invasive blood pressure measuring device and method for operating the non-invasive blood pressure measuring device are provided. The enhanced non-invasive blood pressure measuring device operates by providing audio sensors, ultrasonic sensors, and other sensors, including transmitters, receivers and transducers, to obtain a series of measurements about a subject. The series of measurements may be correlated, such as by machine learning, to extract clinically relevant information.

As disclosed herein, a method for non-invasively monitoring a health condition or pathology based on blood pressure may include obtaining, from the audio transducers, an acoustic signal from a blood vessel. The method may also include converting the acoustic signal to a pressure measurement, the pressure measurement corresponding to blood pressure within the blood vessel. The method may further include sampling the pressure measurement over a sample frequency. The method may in addition include determining average pressure from the sampled pressure measurement over a target time period. The method may further include monitoring the average pressure for deviation from a threshold pressure range. The method may also include generating an alert signal from the blood pressure measuring device if a deviation from the threshold pressure range is detected. Other embodiments of this aspect include corresponding computer systems, apparatus, and computer programs recorded on one or more computer storage devices, each configured to perform the actions of the methods.

Implementations may include one or more of the following features. In some embodiments, the method may include determining that the health condition changed if the deviation from the threshold pressure range is detected. In some examples, the health condition may be a stroke, heart attack, hypertension, hypotension, pulmonary embolism, fibrillation, tachycardia, blood clots, trauma, cancer, kidney disease, respiratory disease, heart disease, thrombosis, thyroid disease, or other health conditions and/or pathologies as known in the art. In some examples, the sample frequency may be between about 50 Hz and about 200 Hz. In some examples, the sample frequency may be between about 25 Hz and 1000 Hz.

The example method may also include determining, with a logical circuit coupled to the audio transducer, detected characteristics of the blood vessel based on the obtained audio signals: identifying, with the logical circuit, the blood vessel as a target blood vessel based on the detected characteristics. The method may also include detecting, with one of the audio transducer, a resonant frequency of the audio signals reflected by the blood vessel, the resonant frequency corresponding to a vibration of a blood vessel wall. The method may also include determining, with the logical circuit, the pressure measurement based on the detected characteristics and the resonant frequency. The method may further include identifying, with the logical circuit, pre-identified detected characteristics and a pre-identified blood pressure measurement corresponding to the target blood vessel; and storing the pressure measurement and the pre-identified detected characteristics corresponding to the target blood vessel are stored in a database.

In some embodiments, the detecting of characteristics of the blood vessel may include determining, with the logical circuit, a type of the blood vessel, where the type of the blood vessel may include a vein, artery, carotid, subclavian, ascending aorta, descending aorta, axillary, brachial, radial, ulnar, palmar arch, renal, iliac, femoral, popliteal, tibial, anterior tibial, dorsalis pedis, posterior tibial, abdominal aorta, genicular, peroneal, plantar/dorsal arch, arcuate, or fibular. In some examples, the method includes determining detected characteristics of the blood vessel by capturing at least one component of the blood vessel including one or more of a wall stiffness, cross sectional diameter, shape, vessel resonance, wall thickness, vessel radius, circumference, clot burden, or vessel plaque thickness of the blood vessel.

The method may further include determining the pressure measurement of the blood vessel by applying, with the logical circuit, the detected characteristics and the resonant frequency to a transformed formula to calculate the blood pressure measurement.

In some examples, the method may also include applying an electronic low-pass filter to remove high frequency pressure artifacts. For example, the high frequency pressure artifacts may include blood pressure fluctuations caused by respiration and/or reflections from blood vessel valves and size variations. Implementations of the described techniques may include hardware, a method or process, or a computer tangible medium.

In one general aspect, the disclosed system may include a plurality of audio transducers configured to capture tomographical information of a physiological structure. The system may also include an audio coupling medium on each of the plurality of audio transducer. The system may furthermore include one or more processors configured to: cause one of the plurality of audio transducers to obtain audio signals reflected by a blood vessel: determine detected characteristics of the blood vessel based on the obtained audio signals: detect, with one of the plurality of audio transducers, a resonant frequency of the audio signals reflected by the blood vessel, the resonant frequency corresponding to a vibration of a blood vessel wall; determine a pressure measurement of the blood vessel based on the detected characteristics and the resonant frequency: sample the pressure measurement over a sample frequency: determine an average pressure from the sampled pressure measurement over a target time period: monitor the average pressure for deviation from a threshold pressure range; and generate an alert signal from the blood pressure measuring device if a deviation from the threshold pressure range is detected. The system may be configured to perform the operations of the methods disclosed herein. Other embodiments of this aspect include corresponding computer systems, apparatus, and computer programs recorded on one or more computer storage devices, each configured to perform the actions of the methods.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates an example computing environment of a measuring device in accordance with some embodiments.

FIG. 2 illustrates an example acoustic sensor or transducer that may be used in the measuring device in accordance with some embodiments.

FIG. 3 illustrates an example computing environment of a measuring device comprising one or more components in accordance with some embodiments.

FIG. 4 illustrates an example process of a measuring device in accordance with some embodiments.

FIG. 5 illustrates an example image of an internal part of a physiological structure that is being monitored by the measuring device in accordance with some embodiments.

FIG. 6 illustrates an example image of a physiological structure with various internal parts of the human body that may be identified and monitored using the measuring device in accordance with some embodiments.

FIG. 7A is a flow chart of an example method for monitoring blood pressure to determine and/or monitor for changes in individual health conditions and/or pathologies.

FIG. 7B illustrates example images of an internal part of a physiological structure that are generated by the measuring device in accordance with some embodiments.

FIG. 8 illustrates an example chip set that can be utilized in implementing architectures and methods in accordance with various implementations of the disclosure.

FIG. 9 illustrates an example methodology for generating a blood pressure measurement as a function of a determined resonant frequency of vibration of a wall of a blood vessel, in accordance with various embodiments of the presently disclosed technology.

FIG. 10 is a companion figure to FIG. 9 that depicts how the methodology of FIG. 9 may be implemented using a computing component, in accordance with various embodiments of the presently disclosed technology.

FIG. 11 illustrates another example methodology for generating a blood pressure measurement as a function of a determined resonant frequency of vibration of a wall of a blood vessel, in accordance with various embodiments of the presently disclosed technology.

FIG. 12 is a companion figure to FIG. 11 that depicts how the methodology of FIG. 11 may be implemented using a computing component, in accordance with various embodiments of the presently disclosed technology.

FIG. 13 illustrates another example methodology for generating a blood pressure measurement as a function of a determined resonant frequency of vibration of a wall of a blood vessel, in accordance with various embodiments of the presently disclosed technology.

FIG. 14 is a companion figure to FIG. 13 that depicts how the methodology of FIG. 13 may be implemented using a computing component, in accordance with various embodiments of the presently disclosed technology.

FIG. 15 illustrates an example chip set that can be utilized in implementing architectures and methods in accordance with various implementations of the disclosure.

FIGS. 16A-16F are plots of results from an example study demonstrating convergence of Young's modulus for blood vessel walls across different blood vessels for different human subjects in support of multiple embodiments disclosed herein.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including.” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of” or “consisting essentially of” the recited components.

In an aspect, the present disclosure provides a device for continuous blood pressure monitoring, comprising a wearable acoustic unit and a processing unit. The wearable acoustic unit may be configured to or may non-invasively detect an acoustic signal from a blood vessel of a subject. The processing unit may be configured to or may use the acoustic signal to continuously monitor a blood pressure of the subject at an error rate of less than or equal to about two percent.

In another aspect, the present disclosure provides a method for continuous blood pressure monitoring. The method may comprise using a wearable acoustic unit to non-invasively detect an acoustic signal from a blood vessel of a subject and process the acoustic signal to continuously monitor a blood pressure of the subject at an error rate of less than or equal to about two percent.

In one example of vital sign detection and monitoring, a measuring device containing a plurality of audio sensors and a processing device may be used to detect vital signs and internal parts of a physiological structure. Internal parts of a physiological structure may include blood vessels, organs, tissues, bones, muscles, tendons, etc. Internal parts of the physiological structure may also include various pathologies including, but not limited to, a fracture, an abscess, a tumor, cellulitis, stones, etc. The measuring device may further use the low or high frequency sound waves to determine properties of the blood vessel and other internal parts of the physiological structure.

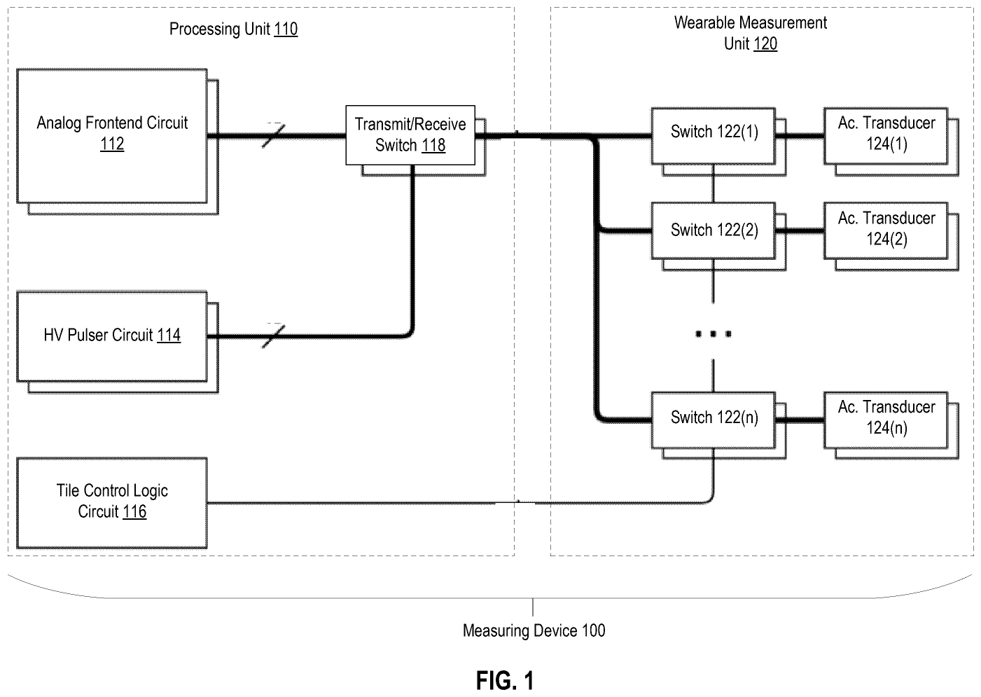

FIG. 1 illustrates an example computing environment of a measuring device that may be affixed to a location of a physiological structure to detect and monitor vital signs and internal parts of the physiological structure. In one embodiment, the measuring device may include an electronic box that comprises the processing device and an armband, wristband, or adhesive patch that comprises the plurality of audio sensors. In another embodiment, the measuring device may include an electronic box that comprises the processing device and at least one audio sensor. The processing device may include an analog frontend, a high voltage (HV) pulser, a transmit/receive switch, and a tile control logic. The armband, wristband, or adhesive patch may include the plurality of audio sensors with low pixel-count arrays for each audio sensor.

For example, as depicted in FIG. 1, measuring device 100 may include a processing unit 110 and a wearable measurement unit 120 (e.g., an adhesive patch, a wearable cuff such as an armband or wristband, etc.). As depicted, the wearable measurement unit may comprise a plurality of acoustic transducers (i.e., acoustic transducers 124(1)-(n)). In certain embodiments, measuring device 100 may comprise a single/unitary physical device comprising processing unit 110 and wearable measurement unit 120. In other embodiments processing unit 110 and wearable measurement unit 120 may be separate physical devices in operative communication with each other.

As depicted, processing unit 110 may comprise an analog frontend circuit 112, a high voltage (HV) pulser circuit 114, a tile control logic circuit 116, and a transmit/receive switch 118. HV pulser circuit 114 can generate audio signals (e.g., high frequency sound waves) of different waveforms and frequencies. Analog frontend circuit 112 can amplify the audio signals generated by HV pulser circuit 114. Tile control logic circuit 116 can work in combination with transmit/receive switch 118 and switches 122(1)-(n) (of wearable measurement device 120) to effectively multiplex the audio signals generated and amplified by analog frontend circuit 112, a high voltage (HV) pulser circuit 114 respectively.

As depicted, wearable measuring unit 120 comprises switches 122(1)-(n) and acoustic transducers 121(1)-(n). In certain embodiments, each of acoustic transducers 124(1)-(n) may comprise low pixel-count arrays for transmitting and/or receiving acoustic energy.

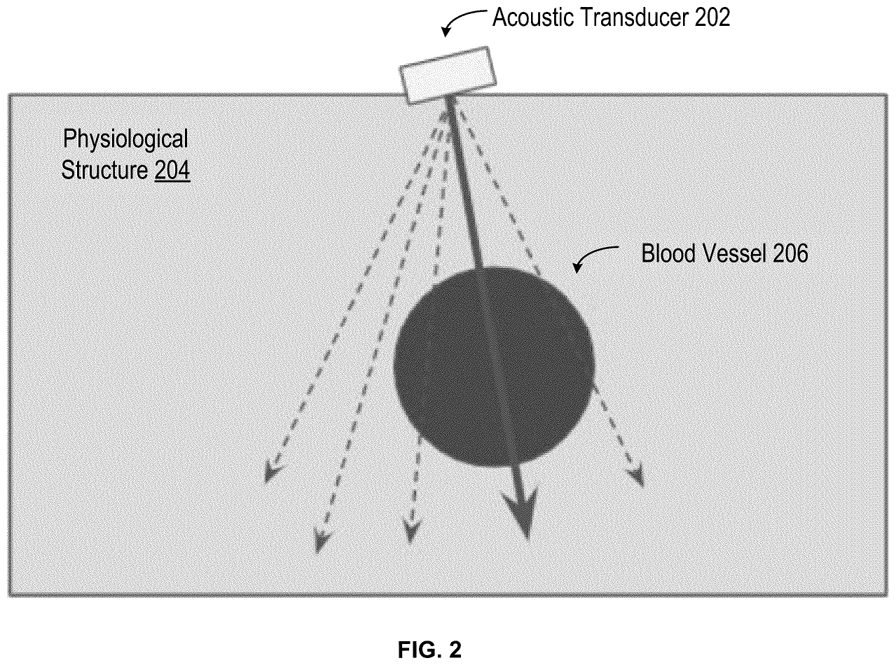

FIG. 2 illustrates an example acoustic sensor and/or transducer that may be used in the measuring device to transmit high frequency sound waves. For example, acoustic sensor and/or transducer 202 that may be used in a measuring device of the presently disclosed technology to transmit and/or receiver acoustic energy (e.g., high frequency sound waves). As depicted, acoustic transducer 202 can transmit audio energy (sometimes referred to herein as acoustic energy) towards a blood vessel 206 through a physiological structure 204 (e.g., human tissue). Generally, a plurality of audio sensors are configured to capture tomographical information of a physiological structure. An audio sensor may be an audio transducer, audio receiver, or audio transmitter. An audio sensor may be used to detect audio energy by transmitting and/or receiving high frequency sound waves. Types of audio energy may include sound, ultrasound, and sonar. The high frequency sound waves may be at different frequencies and produce different pitches and sounds according to the frequency. Different frequencies of high frequency sound waves may help obtain data on different vital signs and internal parts of a physiological structure. Each audio sensor may be set to transmit and/or receive sound waves at different frequencies. With each audio sensor transmitting and/or receiving different frequencies of sound waves, the measuring device may easily generate, detect, and monitor various frequencies of high frequency sound waves to obtain all of the vital signs and internal parts of the physiological structure. Each of the plurality of audio sensors in the armband, wristband, or adhesive patch may be proportionately spaced apart to maximize the output of transmitting and receiving sound waves, thus maximizing the detection and monitoring of vital signs and internal parts of a physiological structure.

Using the audio sensors, either in combination or separately from other components, including low pixel-count arrays, such as 32 elements per audio sensor, phased-array technology and beamforming techniques, the measuring device may collect data of overlapping volumes of a physiological structure. The measuring device may use the collected data to determine measurements associated with vital signs and internal parts of the physiological structure. A vital sign may include blood pressure, heart rate, temperature, respiratory rate, oxygen saturation, cardiac output, stroke volume, pulse, etc. An internal part of the physiological structure may include a blood vessel, artery, vein, organ (i.e., heart, lungs, liver, kidneys, small intestine, large intestine, stomach, brain, etc.), bone, tissue, muscle, tendon, etc., and the internal part may be within the overlapping volumes of the physiological structure. An internal part of the physiological structure may also include various pathologies including, but not limited to, a fracture, an abscess, a tumor, cellulitis, stones, etc. Measurements of a blood vessel may include the blood vessel's dimensions, i.e., blood vessel wall stiffness, blood vessel wall thickness, vessel radius, cross sectional diameter, intima-media thickness, shape, etc., and attributes of the blood vessel, including resonance response of the blood vessel, the velocity of blood flow in the blood vessel, distance between the blood vessel and the measuring device, and plaque thickness in the blood vessel. The measuring device may apply a transformed Laplace's method to obtain measurements of a blood vessel. The measuring device may also use the collected data to produce images of a blood vessel and/or other internal parts within the overlapping volumes of the physiological structure.

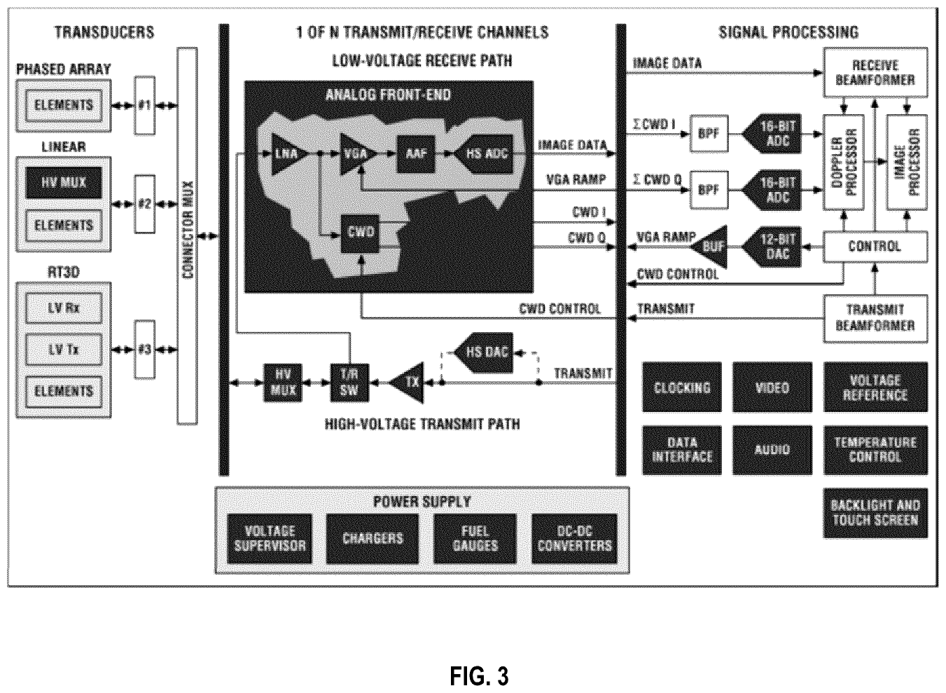

FIG. 3 illustrates an example computing environment of a measuring device comprising one or more components in accordance with some embodiments. In FIG. 3, the audio sensors of the measuring device communicate with the transmit/receive channels. The audio sensors are used to transmit and receive audio energy, such as high frequency sound waves, into a physiological structure. Each audio sensor may be programmed to transmit and/or obtain audio energy at a different frequency. The audio sensors may detect information from the high frequency sound waves that are received. The information detected from the audio sensors are processed through the transmit/receive channels to the signal processing system of the processing device of the measuring device. The signal processing system is used to determine the high frequency sound waves to be transmitted by the audio sensors, and also the high frequency sound waves that are received by the audio sensors. The signal processing system may further process and analyze the received high frequency sound waves to extract data associated with the vital signs and internal parts of the physiological structure that the measuring device is placed against. The signal processing system may process and analyze the received high frequency sound waves by measuring the frequency of the received high frequency sound waves. The measuring device also includes a power supply that is used to power the measuring device.

The measuring device may also include an accelerometer to detect when a physiological structure has fallen. For example, an elderly person may be susceptible to falling and severely injuring herself after a fall to where the elderly person is unable to help herself. The measuring device may also include additional components that may be used to detect and monitor particular attributes and conditions of a physiological structure. Additional components may be included in a measuring device according to the needs of the physiological structure using the respective measuring device. In this way, a measuring device may be customized to include any and all components that may be necessary to detect and monitor attributes relating to the vital signs, internal parts, and physical health of a physiological structure according to the needs of the physiological structure. The measuring device may also be customized to include any and all components that may be necessary to detect and monitor characteristics of inanimate objects, such as pipes, tanks, etc.

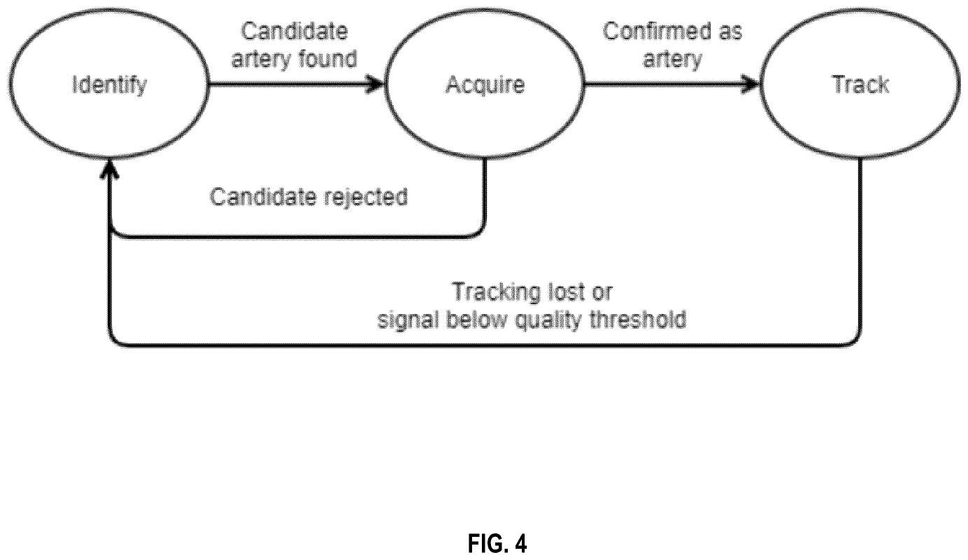

FIG. 4 illustrates an example process of a measuring device. The measuring device may transmit, from the audio sensors, audio energy into a physiological structure. The measuring device may receive, with the audio sensors, audio energy reflected back from internal parts of the physiological structure. The audio energy received with the audio sensors may include information associated to internal parts and vital signs of the physiological structure. The information associated to internal parts and vital signs of the physiological structure may identify any organs or anatomical structures identifiable by audio.

The measuring device may first identify internal parts of a physiological structure. For example, the measuring device may determine if a blood vessel, such as an artery or vein, is found, using the audio energy received by the audio sensors. The audio energy received by the audio sensors may include information associated to a blood vessel in a physiological structure. The information may include data of audio signals reflected by the identified blood vessel. The information may indicate that a blood vessel was found. The measuring device may analyze the information associated to the identified blood vessel to determine vital signs associated with the identified blood vessel. The audio energy received by the audio sensors may include information associated to other internal parts of a physiological structure, including structures surrounding a blood vessel. The information may include data that may be used to assess structures surrounding a blood vessel.

Vital signs associated with a blood vessel may include measurements of the blood vessel and attributes of the blood vessel. Measurements of the blood vessel may include the blood vessel's dimensions, i.e., blood vessel wall stiffness, blood vessel wall thickness, vessel radius, cross sectional diameter, intima-media thickness, shape, circumference, etc. Attributes of the blood vessel man include resonance response of the blood vessel, the velocity of blood flow in the blood vessel, the detection of clots and other particles in the blood vessel, distance between the blood vessel and the measuring device, clot burden and plaque thickness in the blood vessel. The resonance response of the blood vessel may include resonant frequency of audio signals reflected by the blood vessel. The resonant frequency may correspond to a vibration of the blood vessel wall of the blood vessel caused by the reflected audio signals. Resonant frequency in a blood vessel may be used to determine attributes of the artery such as its internal pressure or wall tension. Audio arrays can be used to directly measure diameter of a blood vessel. Analyzing the vital signs of the identified blood vessel may determine the blood vessel type of the identified blood vessel. A blood vessel type may include, but are not limited to, a vein, artery, carotid, subclavian, ascending aorta, descending aorta, axillary, brachial, radial, ulnar, palmar arch, renal, iliac, femoral, popliteal, tibial, anterior tibial, dorsalis pedis, posterior tibial, abdominal aorta, genicular, peroneal, plantar/dorsal arch, arcuate, or fibular. Other arteries not mentioned here may also be considered as types of a blood vessel.

Analyzing the vital signs of the identified blood vessel may also determine if the identified blood vessel is a blood vessel that is desired to be found and monitored, i.e., a target blood vessel. If the measuring device confirms that the correct blood vessel is identified, the measuring device may continuously monitor the blood vessel for a particular duration. The measuring device may use vital signs associated with the identified blood vessel to determine other characteristics of the identified blood vessel.

In one example, the measuring device may apply vital signs, such as a resonant frequency (f), a wall density (p_{S}), a fluid density (p_{L}), a radius-thickness product (y), a wall thickness (h), a blood vessel/arterial radius (a), the arterial wall Young's modulus (E) and the Poisson's ratio of the wall (v), of an identified blood vessel to a transformed Laplace's method that includes one or more transformed formulas, i.e., Equations (1), (2), (3), (4) and (5), to measure blood pressure (P) of the identified blood vessel. Some vital signs, such as resonant frequency (f), wall thickness (h) and blood vessel/arterial radius (a) may be measured by audio imaging from the measuring device. Other vital signs, such as wall density (p_{S}), fluid density (p_{L}) and Poisson's ratio of the wall (v) may be determined from a material database storing measurements of internal parts of physiological structures. Many variations are possible.

The blood vessel/arterial radius (a), resonant frequency (f), Poisson's ratio of the wall (v), wall density (p_{S}), fluid density (p_{L}) and radius-thickness product (y) of the identified blood vessel may first be applied to Equation (1) to determine the arterial wall Young's modulus (E).

$$E = ( \frac{8 {π}^{2} {a}^{7} f ( 1 - {v}^{2} )}{3 γ} ) ( \frac{4 {p}_{L} a ( f + a \frac{d f}{d a} ) + 5 {p}_{S} γ ( \frac{d f}{d a} )}{4 {a}^{7} - 3 {γ}^{2}} ) \qquad Equation ( 1 )$$

The wall thickness (h) and blood vessel/arterial radius (a) of the identified blood vessel may be applied to Equation (2) to determine the parameter α. The parameter α may be a dimensionless parameter that is used to represent the ratio between the wall thickness (h) and blood vessel/arterial radius (a) of the identified blood vessel.

$$α = \frac{h}{a} \qquad Equation ( 2 )$$

The parameter α determined from Equation (2), wall density (p_{S}) and fluid density (p_{L}) of the identified blood vessel may be applied to Equation (3) to determine the parameter p, which may be used to represent units of mass density per unit volume of the identified blood vessel.

$$ρ = α {p}_{S} + \frac{4}{5} {p}_{L} \qquad Equation ( 3 )$$

The arterial wall Young's modulus (E) determined from Equation (1), parameter p determined from Equation (3), resonant frequency (f), blood vessel/arterial radius (a) and Poisson's ratio of the wall (v) may be applied to Equation (4) to determine the parameter D. The parameter D may be a dimensionless parameter that is used to simply the formula, i.e., Equation (5), to measure the blood pressure of the identified blood vessel.

$$D = 4 {π}^{2} ( 1 - {v}^{2} ) \frac{ρ {a}^{2} {f}^{2}}{E} \qquad Equation ( 4 )$$

The blood vessel wall Young's modulus (E) determined from Equation (1), parameter α determined from Equation (2) and parameter D determined from Equation (4) may be applied to Equation (5) to determine the blood pressure (P) of the identified blood vessel.

$$P = ( \frac{9 {α}^{4} - 5 ( 3 α + {α}^{3} ) D + 3 {D}^{2}}{- 4 ( 9 α - {α}^{3} ) + 12 D} ) E \qquad Equation ( 5 )$$

In some embodiments, Young's modulus for a target blood vessel wall may be empirically determined. Although vessel wall stiffness may vary between subjects, using the disclosed technology enables convergence to a unique set of values for pressure and stiffness from any reasonable set of initial conditions.

Understanding the parameters to which the calculated blood pressure is most sensitive is helpful to understanding the accuracy of pressure measurements made using the technology disclosed herein. Sensitivity can be measured using a propagation of error analysis in the equations of the disclosed physical model. Three key understandings related to blood vessel geometry and mechanics underpin this analysis and the physical model of vessel resonance. First, the artery can be modeled as a long cylinder of uniform wall thickness (h) and radius (a). Second, the audio stimulus operates in a perturbative fashion such that any nonlinear effects from the induced vibration can be neglected. Further damping effects due to the finite viscosity of the internal or external media or viscoelasticity of the wall itself can be modeled as a linear effect for the range of displacements induced by the stimulus. And, third, the circumferential Young's Modulus of the arterial wall (E) behaves in a linearly elastic manner in response to the small radius perturbations induced by the stimulus. Such an understanding does not preclude changes in E over the course of a cardiac cycle, only that changes in radius induced by the stimulus are small compared to variations in radius encountered over the course of a heartbeat.

With the above understandings in mind, in one example that implements the disclosed technology, an empirical analysis was performed of convergence at 600 time steps randomly sampled from in vivo carotid measurements (100 from each subject). Studies have estimated circumferential Young's modulus for a carotid artery across multiple healthy adult subjects to vary from ˜0.1 MPa to ˜1 MPa. To account for potential variations due to age or pathologies, the example analysis was extended by a full order of magnitude in either direction, starting with seven initial values for Young's modulus ranging from 0.01 MPa to 10 MPa in geometric steps of 10. A Gauss-Seidel iteration was performed for 5 steps from each starting value, and the seven final results for each of blood pressure (P) and Young's modulus (E) were compiled to compute coefficients of variation (CV), defined as standard deviation divided by mean for each sampled time step. For all 6 subjects, the median CV was less than 0.11% for E and less than 0.01% for P, indicating robust convergence to a unique solution for any reasonable starting value of Young's modulus. Results from the example analysis are provided in the table below:

| Subject | Median Blood Pressure CV | Median Young's Modulus CV |
|---|---|---|
| A | 1.0e−4 | 1.1e−3 |
| B | 5.5e−5 | 5.0e−4 |
| C | 4.3e−5 | 4.9e−4 |
| D | 1.4e−5 | 1.4e−4 |
| E | 2.2e−5 | 2.4e−4 |
| F | 2.8e−5 | 3.9e−4 |

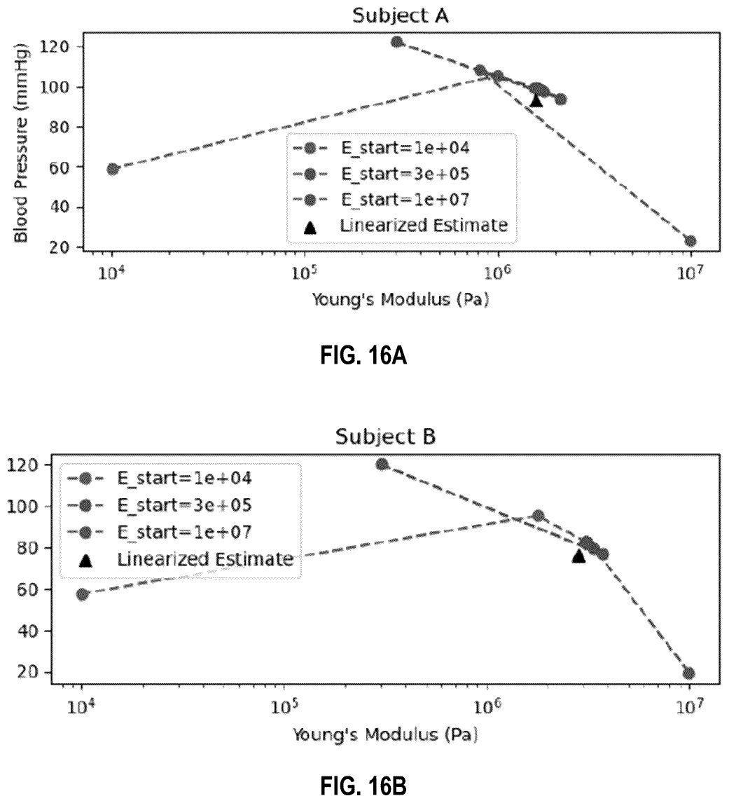

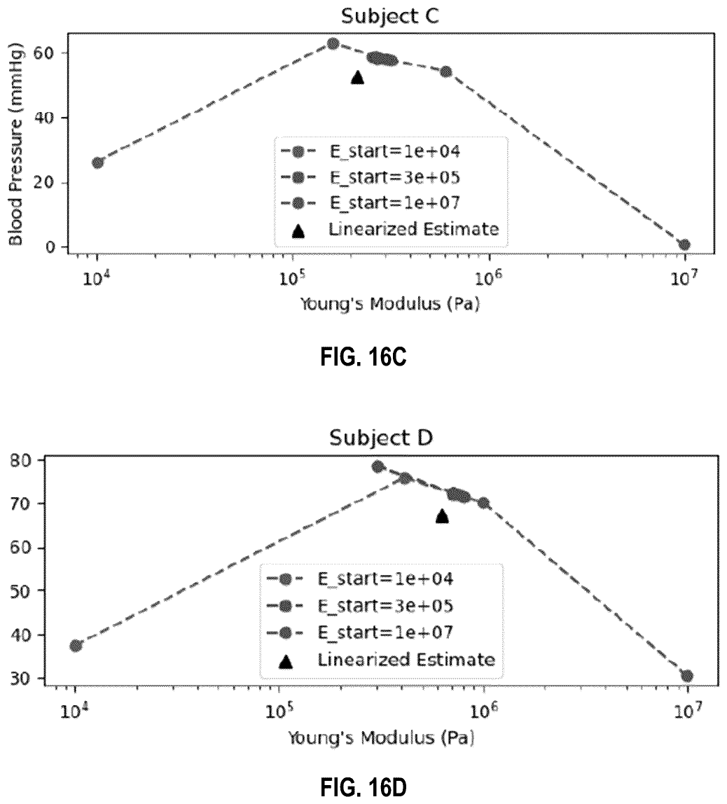

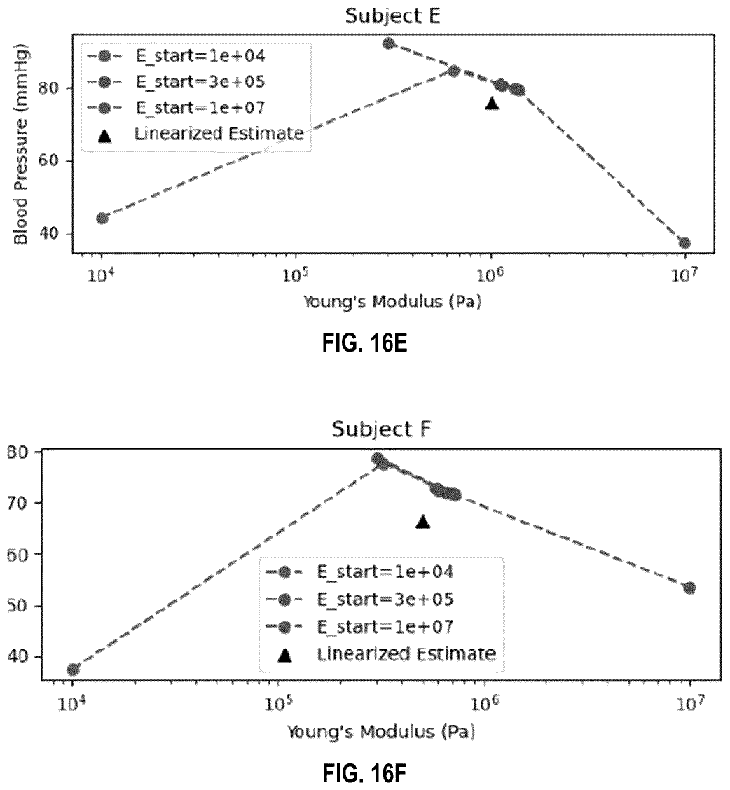

The relationships between blood pressure and Young's modulus from the above subjects are illustrated in FIGS. 16A-F. As illustrated, in each case, the component iterative traces converge to the same location. Continuing with the above-example, Equation 5 may be linearized to obtain a system of linear equations which can be directly solved to find a unique closed-form approximations for P and E. In doing so, it should be noted that it is generally understood that the blood vessel wall is very nearly incompressible. Further, for a fixed length of the vessel wall, its cross-sectional area must remain constant even as pressure changes. It is further understood that the vessel behaves smoothly as radius changes without sharp discontinuities in pressure, stiffness, or frequency. The resulting analysis based on these assumptions reduces to the following equations for P and E:

$$P ( α ) = \frac{5 {π}^{2} f ( 1 - {v}^{2} )}{9} \frac{4 {p}_{L} {α}^{2} ( 4 {α}^{4} f - 5 {γ}^{2} f - 2 {αγ}^{2} \frac{d f}{d a} ) + 5 {ρ}_{S} γ ( 4 {α}^{4} f - 3 {γ}^{2} f - 2 {αγ}^{2} \frac{d f}{d a} )}{4 {α}^{4} - 3 {γ}^{2}} \qquad Equations ( 6 ) and ( 7 )$$

$$E ( α ) = \frac{8 {π}^{2} {α}^{7} f ( 1 - {v}^{2} )}{3 γ} \frac{4 {p}_{L} α ( f + α \frac{d f}{d a} ) + 5 {ρ}_{S} γ \frac{d f}{d a}}{4 {α}^{4} - 3 {γ}^{2}}$$

These equations yield estimates for P and E which are fairly close to their true values. A sensitivity analysis of the above-described relationships reveals that the accuracy of pressure calculations is relatively insensitive to any uncertainty in the wall stiffness, and when combined with uncertainty in blood density, yields an error rate of less than 2% in blood pressure calculations using the methods disclosed herein. In some examples, the measuring device may measure blood pressure at an error rate of less than or equal to about 8%, 6%, 5%, 4%, 3%, 2%, 1%, or less.

The measuring device may detect systolic blood pressure at an accuracy of within about 20 millimeters mercury (mmHg), 18 mmHg, 16 mmHg, 14 mmHg, 12 mmHg, 10 mmHg, 8 mmHg, 6 mmHg, 5 mmHg, 4 mmHg, 3 mmHg, 2 mmHg, or less mmHg of a cuff based blood pressure measurement. In an example, the measuring devices detects systolic blood pressure at an accuracy of within about 8 mmHg of a cuff based blood pressure measurement. In an example, the measuring devices detects systolic blood pressure at an accuracy of within about 5 mmHg of a cuff based blood pressure measurement. The measuring device may detect diastolic blood pressure at an accuracy of within about 20 mmHg. 18 mmHg. 16 mmHg. 14 mmHg. 12 mmHg. 10 mmHg. 8 mmHg. 6 mmHg. 5 mmHg. 4 mmHg. 3 mmHg. 2 mmHg. or less mmHg of a cuff based blood pressure measurement. In an example, the measuring devices detects diastolic blood pressure at an accuracy of within about 8 mmHg of a cuff based blood pressure measurement. In an example, the measuring devices detects diastolic blood pressure at an accuracy of within about 5 mmHg of a cuff based blood pressure measurement. The measuring device may detect pulse pressure at an accuracy of within about 20 mmHg, 18 mmHg, 16 mmHg, 14 mmHg, 12 mmHg, 10 mmHg, 8 mmHg, 6 mmHg, 5 mmHg, 4 mmHg, 3 mmHg, 2 mmHg, or less mmHg of a cuff based blood pressure measurement. In an example, the measuring devices detects pulse pressure at an accuracy of within about 8 mmHg of a cuff based blood pressure measurement. In an example, the measuring devices detects pulse pressure at an accuracy of within about 5 mmHg of a cuff based blood pressure measurement. The measuring device may measure systolic blood pressure at a precision of within about 15 mmHg, 12 mmHg, 10 mmHg, 8 mmHg, 6 mmHg, 5 mmHg, 4 mmHg, 3 mmHg, 2 mmHg, or less. In an example, the measuring device measures systolic blood pressure at a precision of within about 10 mmHg. In an example the measuring device measures systolic blood pressure at a precision of within about 5 mmHg. The measuring device may measure diastolic blood pressure at a precision of within about 15 mmHg. 12 mmHg. 10 mmHg. 8 mmHg. 6 mmHg. 5 mmHg. 4 mmHg. 3 mmHg. 2 mmHg. or less. In an example, the measuring device measures diastolic blood pressure at a precision of within about 10 mmHg. In an example the measuring device measures diastolic blood pressure at a precision of within about 5 mmHg. The measuring device may measure pulse pressure at a precision of within about 15 mmHg, 12 mmHg, 10 mmHg, 8 mmHg, 6 mmHg, 5 mmHg, 4 mmHg, 3 mmHg, 2 mmHg, or less. In an example, the measuring device measures pulse pressure at a precision of within about 10 mmHg. In an example the measuring device measures pulse pressure at a precision of within about 5 mmHg.

The measuring device may also transmit, from the audio sensors, audio energy into an inanimate object. The measuring device may receive, with the audio sensors, audio energy reflected back from internal parts of the inanimate object. The measuring device may receive, with the audio sensors, audio energy reflected back from internal parts of the inanimate object. The audio energy received with the audio sensors may include information associated to internal parts of the inanimate object. The information associated to internal parts of the inanimate object may identify structures and characteristics of the inanimate object that are identifiable by audio.

In one example, the measuring device may determine the blood pressure of a blood vessel. To determine the blood pressure of a blood vessel, the measuring device may be placed against a physiological structure, such as a person, inanimate structure, or animal. The measuring device may be placed against any location of a physiological structure, such as an arm, leg, waist, abdomen, etc., that may contain a blood vessel. In one embodiment, the measuring device may be placed against the upper arm of a physiological structure to detect the blood pressure of the brachial artery. In one embodiment, the measuring device may be placed against the forearm of a physiological structure to detect the blood pressure of the radial artery. In another embodiment, the measuring device may be placed against the thigh/groin/pelvis of a physiological structure to detect the blood pressure of the femoral artery. In another embodiment, the measuring device may be placed against the wrist of a physiological structure to detect the blood pressure of the ulnar or radial artery. In another embodiment, the measuring device may be placed against the abdomen of a physiological structure to detect the blood pressure of the aorta. In another embodiment, the measuring device may be placed against the neck of a physiological structure to detect the blood pressure of a carotid artery. In another embodiment, the measuring device may be placed against the abdomen of a physiological structure, such as a human infant, to detect the blood pressure of the abdominal aorta. In another embodiment, the measuring device may be placed against the wall of a cylindrical thin shell, such as a rocket, to assess pressures of various internal fluids. In another embodiment, the measuring device may be placed against the wall of a cylindrical thin shell such as a pipe to assess internal water or oil pressures.

The measuring device may use the information associated to the vital signs, blood pressure and other characteristics of an internal part, such as a blood vessel, to determine if medications should be made for the physiological structure. The measuring device may determine a medication needs to be made for the physiological structure based on the information associated to the vital signs, blood pressure and other characteristics of an internal part that is currently identified from the audio sensors. The measuring device may determine a medication needs to be made for the physiological structure by comparing information currently obtained from the audio sensors to information previously obtained and/or stored associated to the same internal part.

The measuring device may obtain pre-determined information associated to the vital signs, blood pressure and other characteristics of an internal part, such as a blood vessel. The measuring device may compare the pre-determined information associated to the internal part with presently determined information of the internal part. By comparing the pre-determined information with the presently determined information of the internal part, the measuring device may determine any variations in the vital signs, blood pressure and other characteristics of the internal part.

The measuring device may display information associated to an internal part, such as a blood vessel, on a graphical user interface (GUI). A GUI may include a monitor, screen, projection, television, etc. In one example, the measuring device may display presently determined information of vital signs, blood pressure and other characteristics associated to a blood vessel on a GUI for an individual to view. In another example, the measuring device may display both presently determined information and pre-determined information associated to a blood vessel on a GUI to display any differences between the information.

In one example, the measuring device may determine the current blood pressure of a blood vessel using information associated to the vital signs and other characteristics of the blood vessel that are obtained from audio energy received by the audio sensors. The measuring device may compare the current data of blood pressure, vital signs and other characteristics of the blood vessel to pre-determined data of blood pressure, vital signs and other characteristics of the blood vessel. When the measuring device determines there is a variation between the current data and the pre-determined data of the blood vessel, such as a variation in the blood pressure, the measuring device may determine a medication needs to be made for the physiological structure, such as a person. When the measuring device determines a medication needs to be made for the physiological structure, the measuring device may generate a report. The report may include information associated to the blood vessel, including the current vital signs, blood pressure and other characteristics of the blood vessel. The report may include a medication prescription for the determined medication. The report may be sent automatically to authorized individuals, such as doctors, nurses, medical practitioners, etc. The report may be sent automatically to a system that is used to monitor the health of the physiological structure and process medications for the physiological structure.

In one example, the measuring device may determine that the current value of blood pressure of a blood vessel of a person is above the pre-determined value of blood pressure of the blood vessel of the person. The measuring device may determine a medication needs to be made for the person based on the determination of the difference between the current value and pre-determined value of blood pressure of the blood vessel. The measuring device may then generate a medication prescription for the person. The medication prescription may include information of the variation in the blood pressure of the blood vessel of the person and the type of medication that needs to be made.

In another example, the measuring device may be placed against a physiological structure, such as a person, inanimate structure, or animal, to monitor and assess an organ, such as the heart, lungs, kidneys and liver, of the physiological structure. The measuring device may be placed against a particular location of the physiological structure, such as the chest, abdomen, back, etc., depending on the organ to be monitored. In one embodiment, the measuring device may be placed against the chest of the physiological structure to monitor the heart and assess for any pathology with the heart. In another embodiment, the measuring device may be placed against the back of the physiological structure to monitor the lungs and assess for any pathology with the lungs. In another embodiment, the measuring device may be placed against the abdomen of the physiological structure to monitor the liver and assess for any pathology with the liver.

The measuring device may be attached to an accessory that can allow the measuring device to be placed against a physiological structure and remain in a particular location. The accessory to attach to the measuring device may be an elastic material, such as a strap or band, that can be used to wrap the measuring device against a location of the physiological structure and hold the measuring device in place. The accessory may also be an adhesive material, such as a sticker or adhesive patch, to stick the measuring device against a particular location of the physiological structure and hold the measuring device in place. Keeping the measuring device in the same position against the physiological structure may allow the measuring device to more accurately detect the blood pressure of a blood vessel.

The measuring device may monitor the vital signs and internal parts of a physiological structure until the measuring device is no longer placed against the physiological structure. The measuring device may monitor the vital signs and internal parts of a physiological structure until the measuring device loses power or loses signal in detecting the high frequency sound waves. The measuring device may monitor the vital signs and internal parts of a physiological structure until the measuring device is moved to a location on the physiological structure that is unable to determine and monitor the particular vital signs and/or internal parts of the physiological structure.

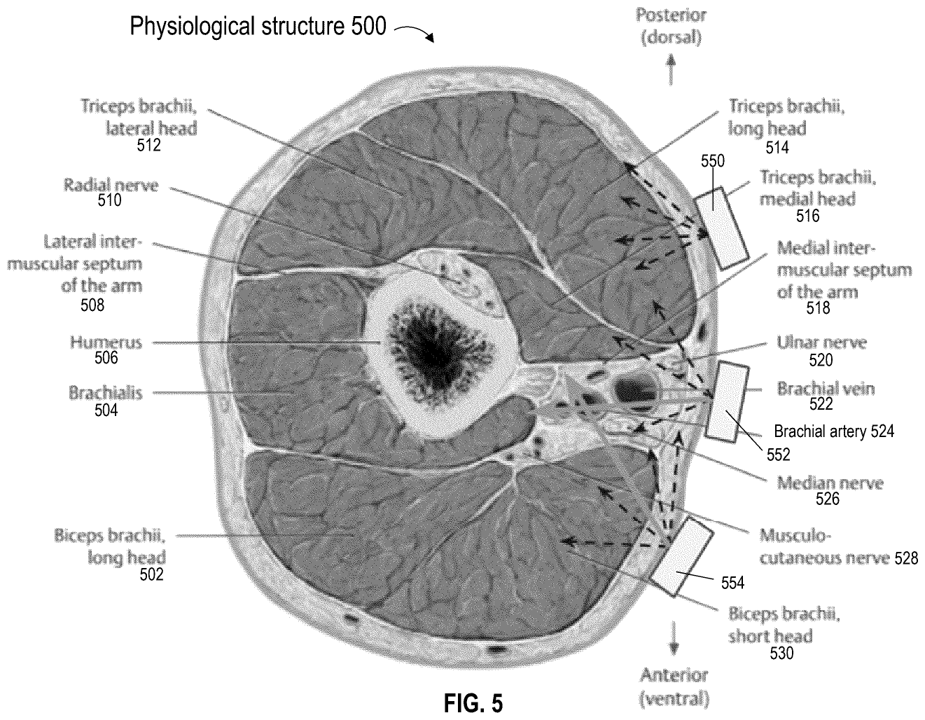

FIG. 5 illustrates an example image of a physiological structure 500 that is being monitored by the acoustic transducers 550, 552, and 554. Acoustic transducers 550-554 may be part of a measuring device of the presently disclosed technology. For example, acoustic transducers 550-554 may be implemented on a wearable arm band or cuff of the measuring device.

As depicted, physiological structure 500 comprises a cross-sectional view of a person's arm. Physiological structure 500 comprises various internal physiological sub-structures which can be monitored/detected by the measuring device. Such internal physiological sub-structures include a biceps brachii (long head) 502, a brachialis 504, a humerus 506, a lateral inter-muscular septum of the arm 508, a radial nerve 510, a triceps brachii (lateral head 512), a triceps brachii (long head) 514, a triceps brachii (medial head) 516, a medial inter-muscular septum of the arm 518, an ulnar 520, a brachial vein 522, a brachial artery 524, a median nerve 526, a musculocutaneous nerve 528, a biceps brachii (short head) 530, etc.

In FIG. 5, the measuring device may be placed at a location of physiological structure 500 where the acoustic transducers of the measuring device (e.g., acoustic transducers 550, 552, and 554) are pressed flat against the surface of the physiological structure. The acoustic transducers may transmit low or high frequency sound or audio waves into physiological structure 500 in multiple directions. The measuring device may use the low or high frequency sound or audio waves to detect and identify vital signs and internal physiological sub-structures of physiological structure 500. As alluded to above, internal physiological sub-structures may include blood vessels (i.e., veins and arteries), organs (i.e., heart, lungs, liver, kidneys, small intestine, large intestine, stomach, brain, etc.), bones, tissues, muscles, tendons, etc. Internal parts may also include various pathologies including, but not limited to, a fracture, an abscess, a tumor, cellulitis, stones, etc. The measuring device may also use the low or high frequency sound waves to measure, induce, or detect frequency response in a blood vessel. The measuring device may further use the low or high frequency sound waves to determine properties of the blood vessel and other internal parts of the physiological structure. The measuring device may apply a transformed Laplace's method or directly image through audio to measure the frequency in a blood vessel to determine attributes of the blood vessel, such as its diameter.

To monitor vital signs and internal parts of a physiological structure, the measuring device may first generate a machine learning (ML) algorithm. The ML algorithm may be generated using initially determined vital signs and other characteristics of internal parts of a physiological structure. The initial vital signs of internal parts may be determined from information associated with the audio energy received by the acoustic transducers. The other characteristics of internal parts may be determined from the initial vital signs. Once the ML algorithm is generated, newly gathered information associated to vital signs and internal parts may be applied to the ML algorithm to determine if any changes have occurred with the vital signs and internal parts of the physiological structure.

In one example, the measuring device may monitor the blood pressure of a blood vessel in a physiological structure, a ML algorithm may be generated using initially determined or pre-determined vital signs and other characteristics of the blood vessel. The initially determined vital signs and other characteristics of the blood vessel may be determined by the measuring device from the audio energy and audio signals received by the acoustic transducers that initially identified the blood vessel. The pre-determined vital signs and other characteristics of the blood vessel may be stored in a database of a system for the measuring device to extract. When the measuring device obtains new vital signs and other characteristics of the blood vessel, the new vital signs and characteristics may be applied to the ML algorithm to determine if any changes have occurred with the vital signs and other characteristics of the blood vessel, including the blood pressure of the blood vessel. Determining changes in the vital signs and other characteristics of internal parts, such as a blood vessel, of a physiological structure may detect and diagnose a medical condition present in the physiological structure.

Health conditions that may be detected and/or monitored in a physiological structure by the measuring device include, but are not limited to congenital cardiac disorder, limb ischemia, cardiovascular anomalies, preeclampsia, sepsis, infection of continuous temp, hypoxia, pneumonia, intubation, complications with pulse oximetry, tachycardia, hypotension, hypertension, internal bleeding, hemorrhage, chronic lung disease, risk of seizures, sleep apnea, postural orthostatic tachycardia syndrome, low blood pressure, low glucose level, deep vein thrombosis (DVT), stroke, pulmonary embolism (PE), superficial thrombophlebitis, blood clotting, tension pneumothorax, supraventricular tachycardia (SVT), idiopathic intermittent atrial fibrillation, angina, myocardial infarction (MI), hyperglycemia, diabetic ketoacidosis (DKT), or cancer.

Methods described herein may further comprise monitoring vital signs of a subject over a period of time. The period of time may be at least about 1 day, 2 days, 3 days. 4 days. 5 days, 10 days, 15 days, 20 days, 30 days, 60 days, 90 days, 120 days, 180 days, 1 year, or more. In an example, methods described herein may comprise monitoring blood pressure of a subject for a period of time of at least about 1 day, 2 days, 3 days, 4 days, 5 days, 10 days, 15 days, 20 days, 30 days, 60 days, 90 days, 120 days, 180 days, 1 year, or more. The method may further include monitoring vital signs, such as blood pressure, pulse rate, oxygenation, or any other vital sign described elsewhere herein, to determine longitudinal trends in vital signs or vital sign data. Longitudinal trends in vital signs or vital sign data may comprise patterns or changes in data collected repeatedly from the subject over the period of time. Determining and monitoring longitudinal trends may provide information related to health status, disease progression, treatment outcomes, or any combination thereof. Health status may include the physical well-being, mental well-being, social well-being, or any combination thereof. In an example, health status is the physical well-being of the subject. Physical well-being of the subject may include the physical health of the subject, such as whether the subject has any biological or physiological dysfunction, symptoms, or other functional impairment. Physical health of the subject may further include whether the subject has a disease, condition, illness, or any combination thereof. The longitudinal trends or data may be generated using artificial intelligence. In an example, the longitudinal trends or data may be generated using machine learning.

The methods described herein may further comprise comparing longitudinal trends or data of the subject with longitudinal trends or data of other subjects. In an example, the longitudinal trends in blood pressure data of the subject may be compared to longitudinal trends in blood pressure of other subjects to generate a comparison of longitudinal trends or data. The comparison of longitudinal trends or data may be generated by comparing longitudinal trends or data of the subject with longitudinal trends or data of other subjects with identified or diagnosed health statuses, disease states, disease progressions, treatment outcomes, or any combination thereof. The longitudinal trends or data of the subject may be compared to longitudinal trends of other subjects using artificial intelligence. In an example, the longitudinal trends or data of the subject may be compared to longitudinal trends of other subjects using machine learning. The comparison of longitudinal trends in vital signs (e.g., blood pressure, temperature, heart rate, oxygenation, etc.) may be usable to determine a health status, predicted disease progress, predicted treatment outcome, or any combination thereof of the subject. Artificial intelligence, such as machine learning, may be used to analyze the longitudinal trends of the subject and/or comparison of longitudinal trends of the subject with longitudinal trends of other subjects to determine the health status, predicted disease progression, predicted treatment outcome, or any combination thereof of the subject. A treatment plan or treatment regimen of the subject may be started, stopped, or modified based at least in part on the health status, predicted disease state, predicted disease progression, predicted treatment outcome, or any combination thereof determined from the longitudinal trends or data of the subject's vital signs. For example, changes in blood pressure over time may indicate a higher risk of cardiovascular events such as sudden cardiac death, heart failure, or stroke. In another example, monitoring longitudinal trends in blood pressure may permit for the prediction of future health risks, early detection and diagnosis of a disease state, monitoring of treatment effectiveness, or any combination thereof.

Determining the health status, predicted disease state, predicted disease progression, predicted treatment outcomes, or any combination thereof may further comprise comparing and analyzing other health related data from the subject with health related data of other subjects. The other subjects may have identified or diagnosed health statuses, disease progressions, treatment outcomes, or any combination thereof. Health related data may include any information related to the subject's health status, such as clinical data, genomic data, behavioral and lifestyle data, environmental data, healthcare system utilization data, device-generated data, imaging data, or any combination thereof. Artificial intelligence, such as machine learning, may be used to generate longitudinal trends or data from the measuring device, from other health related data, or a combination thereof. Artificial intelligence may be used to compare and analyze longitudinal trends or data with other health related data from the subject. Artificial intelligence may be used to compare and analyze longitudinal trends or data generated from the subject with data sets (e.g., longitudinal trends or data or other health related data) generated from other subjects to determine the health status, predicted disease state, predicted disease progression, treatment outcomes, or any combination thereof of the subject.

For example, artificial intelligence may be used to analyze longitudinal trends or data in vital signs of a plurality of subjects with identified or diagnosed health statuses, disease state, disease progressions, treatment outcomes, or any combination thereof to generate classifications of a subject's health statuses, disease state, disease progressions, treatment outcomes, or any combination thereof based on the vital sign longitudinal trends or data and/or other health related data. Artificial intelligence may be used to generate and analyze the subject's vital sign longitudinal trends to classify the health status, disease state, disease progression, treatment outcome, or any combination thereof of the subject. Classification of the subject's health status, disease state, disease progression, treatment outcome, or any combination thereof may be useful for determining to start, stop, or modify a treatment regime. In an example, classifying the subject's health status may permit for active management of a treatment regime by providing real-time or near real-time vital sign data and tracking of changes in vital sign data over time and in response to a treatment. In an example, for a subject on a blood pressure control regimen, the subject's blood pressure may be continuously monitored over time. Artificial intelligence may be used to determine and analyze trends in the subject's blood pressure over time with respect to a dose of blood pressure control therapy being provided. Based on analysis of trends, current dosage, and/or data from other subjects, the dose of the blood pressure control therapy may be modified to improve blood pressure management. By continuously monitoring blood pressure and dosage, a subject's blood pressure therapy dosage may be more accurately and more frequently updated than a subject who's blood pressure and vital signs are periodically monitored for treatment effectiveness. Continuously monitoring blood pressure and vital signs may be useful for monitoring and modifying treatments for a variety of chronic diseases, such as hypertension, hypotension, heart failure, kidney disease, stroke, asthma, chronic obstructive pulmonary disease, diabetes, or metabolic syndrome.

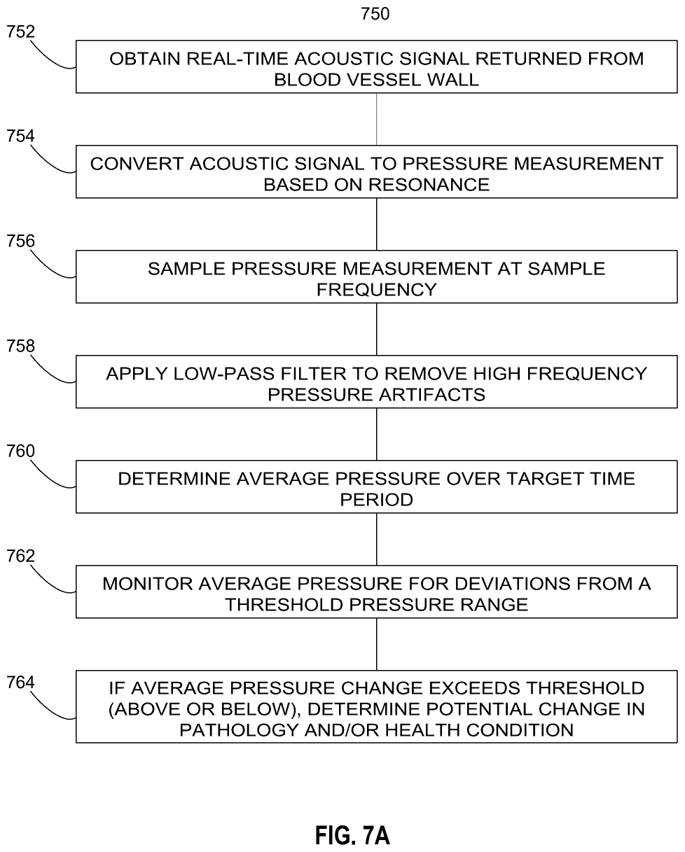

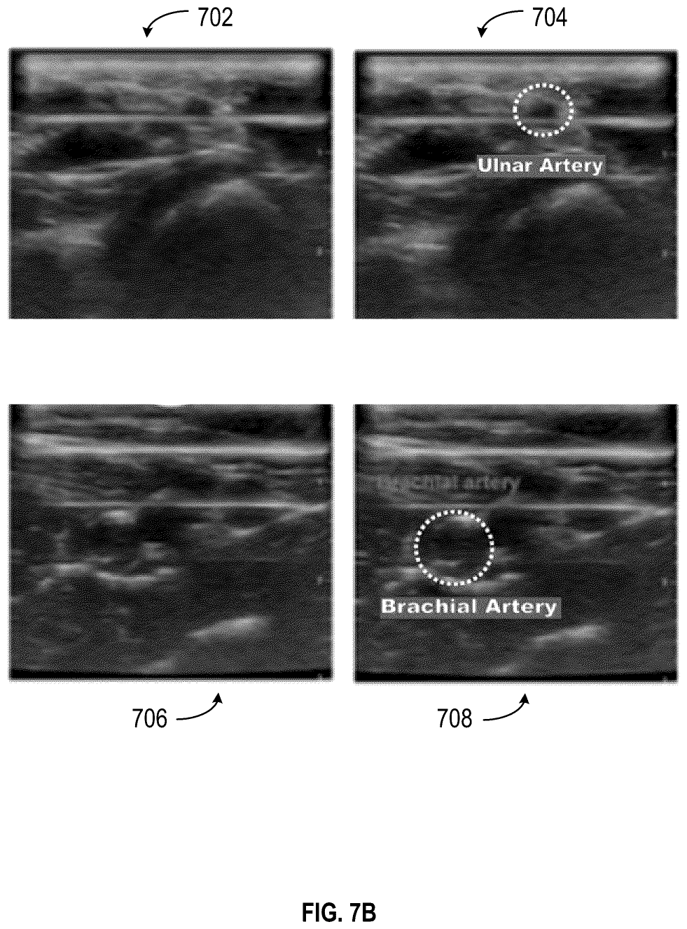

FIG. 7A is a flow chart that illustrates an example method 750 for monitoring blood pressure to determine and/or monitor for changes in pathology and/or health conditions. As shown in FIG. 7A, process 750 may include obtaining, from the audio transducers, an acoustic signal from a blood vessel (block 752). For example, the blood pressure measuring device may obtain, from the audio transducers, an acoustic signal from a blood vessel, as described above. As also shown in FIG. 7A, process 750 may include converting the acoustic signal to a pressure measurement, the pressure measurement corresponding to blood pressure within the blood vessel (block 754). For example, device may convert the acoustic signal to a pressure measurement, the pressure measurement corresponding to blood pressure within the blood vessel, as described above. As further shown in FIG. 7A, process 750 may include sampling the pressure measurement over a sample frequency (block 756). For example, the blood pressure measuring device may sample the pressure measurement over a sample frequency, as described above. Process 750 may include applying an electronic low-pass filter to remove high frequency pressure artifacts (block 758). For example, device may apply an electronic low-pass filter to remove high frequency pressure artifacts, as described above. Process 750 may include determining average pressure from the sampled pressure measurement over a target time period (block 760). For example, the blood pressure measuring device may determine average pressure from the sampled pressure measurement over a target time period, as described above. As also shown in FIG. 7A, process 750 may include monitoring the average pressure for deviation from a threshold pressure range (block 762). For example, the blood pressure measuring device may monitor the average pressure for deviation from a threshold pressure range, as described above. As further shown in FIG. 7A, process 750 may include generating an alert signal from the blood pressure measuring device if a deviation from the threshold pressure range is detected (block 754). For example, the blood pressure measuring device may generate an alert signal from the blood pressure measuring device if a deviation from the threshold pressure range is detected, as described above. The alert signal may be an audible signal, an SMS message, a message in a graphical user interface, a haptic signal, or other type of alert signal. In some examples, the alert signal may include information identifying a change in health condition and/or pathology.

Although FIG. 7A shows example blocks of process 750, in some implementations, process 750 may include additional blocks, fewer blocks, different blocks, or differently arranged blocks than those depicted in FIG. 7A. Additionally, or alternatively, two or more of the blocks of process 750 may be performed in parallel.

In some examples, changes in health conditions and/or pathologies will correlate to changes in blood pressure over time. Methods disclosed herein may include continuous monitoring of blood pressure within a specified blood vessel. Given the high accuracy of blood pressure measurements described herein, small or micro changes of blood pressure may be detected in the blood vessel over small periods of time. In some examples, the sampling frequency for blood pressure measurements may be between about 50 Hz and about 1000 Hz. In some examples, the sampling frequency may be less than about 50 Hz. In some examples, the sampling frequency may be between about 75 Hz and about 200 Hz. In some examples, the sampling frequency may be about 100 Hz. In some examples, the sampling frequency may be about 125 Hz. In some example, the sampling frequency may be about 150 Hz. In some examples, the sampling frequency may be about 175 Hz, 200 Hz, 225 Hz, 250 Hz, 275 Hz, 300 Hz, 325 Hz, 350 Hz, 375 Hz, 400 Hz, 425 Hz, 450 Hz, 475 Hz, 500 Hz, 525 Hz, 550 Hz, 575 Hz, 600 Hz, 625 Hz, 650 Hz, 675 Hz, 700 Hz, 725 Hz, 750 Hz, 775 Hz, 800 Hz, 825 Hz, 850 Hz, 875 Hz, 900 Hz, 925 Hz, 950 Hz, or 975 Hz, or within any of the ranges therein. For purposes of these measurements, the term “about” means plus or minus 10 Hz.

Taking accurate blood pressure measurements in the sampling frequencies described above may also capture changes in blood pressure due to respiration and/or reflections of blood flow from blood vessel valves, occlusions, and/or vessel size changes. These regularly recurring pressure changes may create higher frequency recurring patterns in the blood pressure measurements over time that can be filtered out, for example, using a low-pass filter.

Changes in average blood pressure, after removing high frequency artifacts described above, may then be monitored. Increases in the average blood pressure over thresholds may then be used to determine or monitor for hypertension, stress, blood clots, micro clots, potential stroke, adverse reactions to food or medications, kidney disease, obstructive sleep apnea, thyroid disease, or other potential conditions caused by high blood pressure. In some examples, these changes may be used to trigger corrective action, including taking medication to reduce blood pressure, reduction of salt intake, triggering of exercise, or other activity. In some examples, the blood pressure warning thresholds may be preset by a medical professional to monitor for predetermined conditions for which blood pressure changes may indicate an adverse change in the patient's condition.

Similarly, decreases in the average blood pressure below thresholds may be used to determine and monitor for orthostatic hypotension, postprandial hypotension, neutrally mediated hypotension, multiple symptom atrophy, internal bleeding, or other conditions associated with low blood pressure. In some examples, these changes may be used to trigger corrective action, including taking medication, stopping activity, ingesting fluids, sitting or laying down, or other activity. In some examples, the blood pressure warning thresholds may be preset by a medical professional to monitor for predetermined conditions for which blood pressure changes may indicate an adverse change in the patient's condition.

In some examples, the technology disclosed herein may be used to associate blood pressure changes with respiration to determine and monitor for acute adverse changes in respiration indicative of medical conditions such as heart attack or other acute heart malfunction, lung disease or infection, pulmonary embolisms, and/or trauma. In some embodiments, the blood pressure measuring device will generate an alert to the user and/or health care professional if a health condition and/or medical emergency is determined based on the monitoring conditions described above, including by alerting the user and/or health care professional to abnormal changes in blood pressure.

In some embodiments, the technology disclosed herein may detect micro-changes in average blood pressure on the order of less than 10 mmHg over one or more sampling periods. In some embodiments, long term blood pressure monitoring may be tracked and logged to determine changes in average blood pressure that may be indicative of changing patient pathology. In some embodiments, blood pressure change patterns may be tracked across multiple individuals and/or populations and correlated with health characteristics and pathologies to empirically determine trigger pressure patterns that may be indicative of specific pathologies, diagnoses, health risks, and/or health conditions. In some embodiments, machine learning techniques may be used to analyze training blood pressure data acquired from multiple individuals and or populations to create a continuous blood pressure monitoring model capable of recognizing blood pressure patterns in individual subjects that may be indicative of any of the health conditions and/or pathologies disclosed herein.

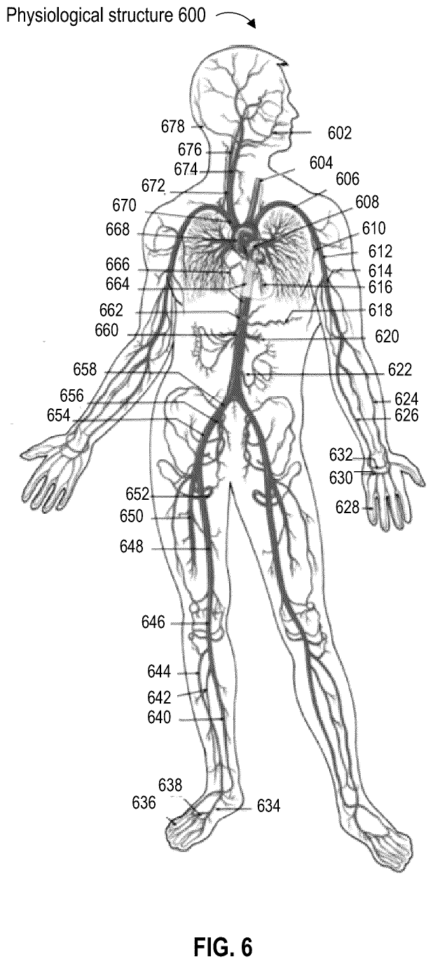

FIG. 6 illustrates an example image of a physiological structure 600 of a human body with various internal physiological sub-structures of the human body that may be identified and monitored using the measuring device. Such internal physiological sub-structures may include internal physiological sub-structures 602-678. As depicted, internal physiological sub-structures 602-678 may include various types of blood vessels. In one example, a blood vessel may include an artery or vein in physiological structure 600. The measuring device disclosed herein may detect pressure from any of the following blood vessels; a vein or an artery (including, e.g., the carotid, subclavian, ascending aorta, descending aorta, axillary, brachial, radial, ulnar, palmar arch, renal, iliac, femoral, popliteal, tibial, anterior tibial, dorsalis pedis, posterior tibial, abdominal aorta, genicular, peroneal, plantar/dorsal arch, arcuate, fibular, or others). The measuring device may be placed against a particular location of physiological structure 600 to detect the blood pressure of a particular blood vessel, based on any number of factors, including the type of physiological structure (e.g., adult person, infant person, adult animal, infant animal, etc.) and the blood vessel of interest. Keeping the measuring device in essentially the same position against physiological structure 600 may allow the measuring device to more accurately detect the blood pressure of the blood vessel of interest.

While very high resolution and/or quality imaging of the internal anatomy, such as a blood vessel, is not necessary for the purpose of detecting and monitoring vital signs of a person, a reasonable quality image may allow for improved and faster identification and measurements of the internal part, depending on the relative size of the blood vessel of interest. The image quality should allow for visualization of the diameter of the blood vessel of interest to enable measurements thereof. An audio coupling medium may be placed on each acoustic transducer. The audio coupling medium may include a pad of a lubricant substance, such as silicone, gel, or hydrogel, which functions as an acoustic-impedance matching layer. The audio coupling medium may allow clearer imaging of an internal part, such as a blood vessel, in comparison to imaging of the blood vessel obtained from using acoustic transducers without an audio coupling medium.

In one example, a blood vessel may include an artery or vein in the physiological structure. A blood vessel whose blood pressure is detected by the measuring device may include a vein, artery, carotid, subclavian, ascending aorta, descending aorta, axillary, brachial, radial, ulnar, palmar arch, renal, iliac, femoral, popliteal, tibial, anterior tibial, dorsalis pedis, posterior tibial, abdominal aorta, genicular, peroneal, plantar/dorsal arch, arcuate, fibular, or others. The measuring device may be placed against a particular location of a physiological structure to detect the blood pressure of a particular blood vessel, based on any number of factors, including the type of physiological structure (i.e., adult person, infant person, adult animal, infant animal, etc.) and the blood vessel of interest. Keeping the measuring device in the same position against the physiological structure may allow the measuring device to more accurately detect the blood pressure of the blood vessel of interest.

FIG. 7B illustrates example images 702, 704, 706, and 708 of an internal part of a physiological structure that are generated by the measuring device. The example images show two sets of images taken at different locations of a physiological structure to detect and monitor different internal parts of the physiological structure. Namely, images 702 and 704 depict an ulnar artery while images 706 and 708 depict a brachial artery. Both these arteries may be detected and monitored by a measuring device of the presently disclosed technology.

Further, the measuring device may use the collected data or pre-identified data of a person's vital signs and internal parts, in combination or separately, with one or more measurements and produced images of the internal parts to generate an algorithm. In one example, the algorithm may be used to determine an accurate location of a blood vessel in the physiological structure. The pre-identified data collected data, measurements, images, and/or location of the blood vessel may be displayed and viewed on a screen. The screen may be on the measuring device and/or on another device that is associated with the measuring device.

After the measuring device has generated an algorithm for a particular internal part, such as a blood vessel, the measuring device may monitor the particular internal part. In one example, a particular blood vessel may be monitored by continuously having the measuring device placed against the physiological structure at a location where the blood vessel is located. In another example, a particular blood vessel may be monitored by periodically placing the measuring device against the physiological structure at a location where the blood vessel is located. By having the measuring device placed against the physiological structure at a location where the particular blood vessel is located, the measuring device may obtain new data on the blood vessel. The measuring device may use the new data to determine any changes to or associated with the particular blood vessel, including any changes to other organs, bones, tissues, etc. of the surrounding physiological structure. In one embodiment, the measuring device may compare the new data against the generated algorithm for the particular blood vessel to determine any changes to the particular blood vessel.

Using the measuring device to monitor vital signs and internal parts, such as blood vessels, for any changes may allow the determination of any issues occurring in the physiological structure. In one embodiment, the measuring device may be used on a person to detect and monitor arterial lines for beat-to-beat monitoring to determine if any changes are occurring with the person's blood pressure, and make adjustments to vasoactive medications, such as a pressor, based on data on the blood pressure. The measuring device may be used in substitution of an arterial catheter in monitoring blood pressure, or be used in combination with an arterial catheter for multiple arterial blood sampling and allow for earlier removal of the arterial catheter when arterial blood samples are no longer needed.

In an embodiment, the measuring device may obtain various vital sign measurements of a person. The measuring device may also be used to continuously obtain vital sign measurements of a person and automatically send the data to associated devices. With the measuring device being able to obtain various vital sign measurements, it may replace the use of multiple devices, such as a blood pressure cuff, electrocardiogram (EKG), and pulse oximeter, where each are needed to measure a single type of vital sign. Being able to use the measuring device in replacement of multiple devices and tools may allow for more ease in obtaining and monitoring measurements of a person and more quickly determine a diagnosis and treatment for the person. Using the measuring device may also allow for accurate, noninvasive readings and monitoring of a person's vital signs, compared to other devices. Using a single measuring device to obtain and monitor various vital signs may be less problematic and allow for obtaining faster results than using multiple different devices and tools, especially in emergency situations, where time is critical to saving a person who is in a life-threatening condition.

The measuring device may be portable and may run on battery, which may be chargeable and/or replaceable. The measuring device may be used in conjunction with other devices, such as computers, monitors, phones, tablets, etc. The measuring device may connect to other devices via a wired or wireless connection, such as Bluetooth. With the measuring device being portable, it may be used to obtain measurements of vital signs and internal parts of a physiological structure in any situation and location.

The measuring device may send an alert when the measuring device is low on battery. The measuring device may send an alert when there is an issue with the communication connection it has with another device. The measuring device may send an alert when there is an issue with a function of the measuring device, such as an issue with the audio sensors. The measuring device may send an alert to another device that it is connect to. The measuring device may send an alert to a system, application, or platform that it is connect to. The measuring device may display an alert on an GUI with a message or symbol indicating any issue that is occurring with the measuring device. Many variations are possible.

In another example, emergency responders, such as paramedics and emergency medical technicians (EMTs), may use the measuring device on a person when in a noisy and chaotic environment, such as the middle of a congested street. The emergency responders may also attach the measuring device to a person using an accessory to the measuring device, such as an elastic band or adhesive patch, allowing the emergency responders to move and transport the person while continuously obtaining measurements. Being able to obtain continuous measurements of a person during emergency situations may allow faster and more accurate diagnosis and treatments to be implemented on the person. In another example, emergency responders will be able to use the measuring device to obtain measurements of an injured person and determine the condition and status of the injured person. Knowing the condition and status of the injured person, the emergency responders may better determine if the injured person healthy enough to be transported to a medical facility, such as a hospital. STEMI receiving center, stroke center, etc. The emergency responders may also be able to provide any preliminary medications and treatments to the injured person before and while transporting the injured person to a medical facility. By continuously monitoring the measurements of the injured person, the emergency responders may be able to update preliminary medications and treatments to the injured person, and update their navigational course.

The measuring device may also provide easier monitoring of individuals during or after a mass casualty incident, such as an accident or natural disaster. An emergency responder may be able to attend to multiple individuals at the same time by using a measuring device for each person. By using a measuring device on each person involved in a mass casualty incident, the emergency responder may attend to one person while still obtaining data and a diagnosis of all of the individuals. The emergency responder will also be able to receive immediate feedback from each measuring device allowing for quicker responses and treatment from the emergency responder. This may allow the emergency responder to determine which person of the multiple individuals needs immediate medical treatment, and may save time and resources by allowing the emergency responder to provide accurate treatments to each person. Each measuring device may also send data, alerts and message, of the respective person it is attached to, to medical personnel and rescuers so that accurate treatment may be provided and at a faster response time. The data, alerts and messages may also provide information of any changes that are occurring with the health of the respective person so that treatment may be updated accordingly.

The measuring device may be used on a physiological structure, such as a person, who needs to have their vital signs continuously monitored. The measuring device may continuously obtain vital sign measurements, such as blood pressure, and other data of a person as the measuring device is attached to the person's person. The measuring device may analyze the vital sign measurements and other data that it obtains from the person. The measuring device may determine any variations in the vital sign measurements and other characteristics of internal parts of a person from analyzing the data that it obtains from the person. The measuring device may be programmed to automatically send data to a doctor, or any other individual associated with the measuring device, or manually chose when to send the data. The measuring device may send all of the obtained and analyzed data, including any variations in the vital sign measurements and other characteristics of internal parts of a person, to a doctor so the doctor may continuously monitor the person's health. The measuring device may be connected to another device, such as a computer, phone, tablet, etc., to send the data to an individual. The measuring device may be connected to a system, application, or platform, such as telemedicine platforms, where the data may be uploaded to a database for other individuals, such as doctors, nurses, medical practitioners, etc., to access. The measuring device may protect and lock the data by assigning a code or password to the data, and provide such code or password to authorized individuals for access. Providing data to authorized individuals, such as doctors and medical personnel, may help with the study of health issues so that improved and accurate diagnosis and treatment may be provided to individuals. The measuring device may automatically upload data to a cloud database of a system to easily transmit information from patients to their clinical care teams, for research applications, or for the patient's personal information and storage.

The measuring device may send alerts, such as vibrations, sounds, messages, etc., and/or messages to the person attached to the measuring device, when the measuring device determines an issue with the person based on the obtained data. The measuring device may also send alerts and messages to other individuals, such as doctors, family members, friends, etc., when the measuring device determines there is an issue with the person based on the data. The alerts and messages may be sent automatically to authorized individuals. There may be a plurality of alerts that may be sent, where each alert includes a particular message. A message may include data of the person, and may include a recommended diagnosis and treatment for the person (e.g. low blood pressure, seek medical advice), based on the data. The measuring device may also provide updates, using alerts and messages, to the person attached to the measuring device, where the updates notify the person of actions the person should take based on the data. Such actions may include visiting a doctor, taking medication, calling for emergency assistance, etc. Alerts and messages sent from the measuring device may help prevent or dissipate medical problems with a person, and help save a person's life when medical assistance is urgently needed.

The measuring device may be used on any individual who needs to be monitored, whether periodically, continuously, or just for a one time occasion. In one example, a person with hypertension may attach and use the measuring device on their person to continuously monitor the person's blood pressure. The measuring device may periodically send data of the person's blood pressure to the person's doctor who has been authorized by the person to access the data. When the measuring device determines, based on the data, that the person's blood pressure is beginning to increase or decrease, the measuring device may send an alert to the person so that the person may implement actions to restabilize their blood pressure. When the measuring device determines, based on the data, that the person is in critical condition and is in need of emergency treatment, the measuring device may send messages to all individuals who have been authorized to receive emergency notifications regarding the person.

In another example, a person with risks for clotting may attach and use the measuring device on their person to continuously monitor the person's vital signs to determine any increased clot burden on the person. The measuring device may also be used on a person who is at risk for deep vein thrombosis (DVT), stroke, pulmonary embolism (PE), apnea, hypotension, hypoxia, and other risks. The measuring device may obtain vital sign data of the person and use the data to assess for microthrombi, blood viscosity, and other metrics. The measuring device may provide feedback, such as recommended diagnosis and treatment, for the patient based on the data assessment. There is no limitation to the type of person or purposes for a person to use the measuring device.

Hospitals and medical personnel may also use the measuring device on patients to monitor their vital signs. Using the measuring device may allow medical personnel to obtain a better understanding of a patient's health and provide better treatment to the patient. In one example, a nurse may use the measuring device on a dialysis patient, who has abnormal blood pressure which can change rapidly during dialysis. The nurse may be able to use the vital sign data of the patient obtained by the measuring device to understand any habits with the patient's vital signs. The nurse may then predict when the patient may have a rapid decrease in blood pressure and adjust the dialysis parameters accordingly to accommodate before the patient experiences symptoms of nausea, dizziness, or syncope. The nurse may also use the measuring device to monitor for any changes in the patient's heart rate, respiratory response, and oxygen saturation (O₂ sat).

Using the measuring device on patients, hospitals and medical personnel may further be able to monitor vital signs of a patient before a procedure is performed on the patient. This may be to ensure that the patient is healthy enough to have the procedure performed on the patient. A patient's health may be determined to be healthy if the measuring device analyzes the data of the patient and determines that the overall health of the patient is above a threshold. The measuring device may also be used on the patient during and after a procedure is performed on the patient to monitor the patient's vital signs to ensure the patient is not experiencing any health issues during and after the procedure. If a health issue is determined by the measuring device, the measuring device may send an alert and message to authorized individuals, such as the patient, medical personnel, family members, etc., to notify them of the issue. This may help with providing accurate treatments to patients and minimalizing any health issues that may occur from performing a procedure on a patient.

The measuring device may be used to determine the health of a person and if the person's health is above a requirement for a particular task or event. In one example, the measuring device may be used on individuals who are traveling long distances, such as a space tourist. In order for a person to be accepted to travel to space as a space tourist, the person may have to have an overall health that is over a threshold. The measuring device may be used to continuously monitor a person for a period of time leading up to space travel, so that administrators may know if the person is healthy enough to be a space tourist upon the day of departure of the space travel. The measuring device may also be able determine if the overall health of a person, based on the data obtained of the person, is above a given threshold to permit the person to join in the space travel. The measuring device may also be used on a person when they are in course of their travels to ensure that any health issues that arise during travel will be found as soon as possible. The measuring device may send an alert and/or message to crew members and/or the ground crew of the flight so that medical attention may be provided to a person experiencing a health issue.

The measuring device may provide an easy means of obtaining vital sign measurements of a person when the person is in various states, such as stressed, relaxed, sleeping, awake, etc. In one example, the measuring device may be used on a person who is easily stressed to monitor the person's vital signs when the person is in various states throughout the day. By continuously monitoring the person's vital signs, the measuring device may accurately diagnosis the person of whether the person has any conditions, such as hypertension. The measuring device may determine that the person only has hypertension when the person is stressed. The measuring device may then send such determination and data of the person to an authorized medical personnel, so the authorized medical personnel may properly diagnose and treat the person. The measuring device may also access the data of vital sign measurements to determine the condition of the person's body, like if the person is overheating, experiencing presyncope, showing signs of illness, fatigue, and the person's overall fitness. This may prevent inaccurate diagnosis and treatments to be provided to a person by the measuring device and medical personnel receiving the data.

The measuring device may be useful to a person in a profession that requires them to be place their body in stressful conditions. In one example, an astronaut may place their body under various stresses, including pressure, atmospheric or temperature changes, when going through launch and reentry of Earth's atmosphere, extravehicular activity (EVA), and periods of spaceflight. The measuring device may enable flight surgeons and other members of an astronaut team to monitor the vital sign measurements of an astronaut during every stage of a space mission to ensure that the astronaut is healthy and not undergoing any issues.

In another example, a soldier may place their body under various stresses when on the battlefield, such as during live ammunition fire. A soldier may include a warfighter, pilot, marine, or any individual serving in a military capacity. The measuring device may monitor vital signs of a soldier and assess injuries that the soldier may have sustained. Based on the data, the measuring device may also send the data, along with alerts and messages, to medical personnel who may attend to the soldier's medical needs. Using the measuring device may help save lives by providing accurate and up-to-date data of a person's health, allowing for accurate diagnosis and treatment to be provided, thus improving assessment and evacuation times. The measuring device may also be used on a soldier during training to determine if the soldier is healthy enough for a particular mission.

The measuring device may provide an easier and more comfortable means in obtaining vital sign measurements of a person who is otherwise unable to do it in the presence of a medical person, such as a doctor. In one example, a person who gets easily stressed and uncomfortable while in the presence of a doctor, or any medical person, may provide difficulty in providing accurate vital sign measurements in the medical person's presence. The measuring device may allow the person to more easily obtain vital sign measurements in a comfortable environment, such as their own home, and may send the data to the medical person or any other authorized individual, or a platform or device. This is particularly relevant to patients with healthcare anxiety or situational/white coat hypertension.

The measuring device may be used by any person who wants to obtain and monitor their vital signs. A person may want to obtain and monitor their vital signs to better understand their body and overall health. A person may want to obtain and monitor their vital signs for a purpose, such as to improve fitness or meet a performance goal. The measuring device may be implemented as any device that can be attached to a person who wants or needs to have their vital signs obtained, measured and monitored. In one example, the measuring device may be implemented as a watch that an athlete may wear on their wrist. The athlete may be training for a competition and may need and want to obtain, measure and monitor their vital signs to help improve and adjust their training. Data obtained from the measuring device may be sent to the athlete's trainers so that they may adjust and improve the athlete's training based on the data. In another example, a mountain climber may need to obtain, measure and monitor their vital signs while climbing a mountain. The measuring device may continuously obtain and monitor a mountain climber's vital signs, and use the obtained data to generate acclimatization protocols for the mountain climber. The measuring device may send alerts and messages to the mountain climber to indicate different actions for the mountain climber to take, such as when to ascend, descend, abort, or remain stationary to obtain optimal acclimatization or avoid conditions such as high altitude pulmonary/cerebral edema.

The measuring device may also be helpful on individuals who are in a remote location where medical diagnosis from a medical person is difficult to obtain. Such remote locations may also include locations that do not have access to modern medicine. In one example, the measuring device may be used on a person who is on an expedition and exploring the rain forest of a tropical island. If the measuring device determines that the explorer is having a health issue, based on data obtained from the explorer, then such data may be sent to associated individuals, such as a search and rescue team, who may come to the explorer's aid. The measuring device may also send alerts and messages to any associated individuals, where the alerts and messages may include a recommended diagnosis and treatment determined by the measuring device based on the data. The data, alerts and messages may provide an associated individual with information needed to provide accurate treatment to the explorer, and make decisions such as whether to request a medical evacuation and what type of transportation to request, such as a plane, helicopter, boat, etc. If more than one explorer is on the same expedition, then associated individuals may know how many individuals are in need of medical attention or have had loss of vital signs.

The measuring device may be associated with a company, such as a health provider. insurance provider, etc. A person who is using the measuring device may link a company to the measuring device. The company may receive notifications from the measuring device, where the notifications may include data associated with the vital sign measurements of the person. The company may provide benefits to the person according to data. In one example, an insurance company may determine from the data that the person is within the 90th percentile in health for individuals of similar attributes, such as age, sex, height, etc. The insurance company may provide a discount in health insurance based on the determination. The insurance company may continue to provide a discount in health insurance to the person as the person continues to be within the 90th percentile in health for a particular group of individuals. The discount provided by the insurance company may vary based on the overall health determination of a person from the person's data.

The measuring device may be used on multiple locations on a physiological structure by placing the measuring device against the desired location of the physiological structure, i.e. arm, leg, waist, hip, neck, etc., where vital signs and internal parts of the physiological structure want to be obtained and monitored. The measuring device may also be attached to any location of the physiological structure using an accessory, such as an adhesive or elastic material, that will keep the measuring device in place at the particular location. The measuring device may be attached to the physiological structure for various periods of time to obtain data on the person's vital signs and internal parts of the person at various times of the day, when the person is under various conditions and when the person is performing various tasks.

Following obtaining and monitoring a physiological structure's vital signs, the measuring device may analyze all of the obtained data of the physiological structure and determine a diagnosis for the physiological structure. Non limiting examples of diagnosis that the measuring device may determine are congenital cardiac disorders, pulses and risks for limb ischemia, cardiovascular anomalies such as aortic dissection and vessel occlusion, preeclampsia, sepsis, infection of continuous temp, hypoxia, pneumonia, intubation, complications with pulse oximetry, tachycardia, hypotension, hypertension, internal bleeding, hemorrhage, chronic lung disease, risk of seizures, sleep apnea, postural orthostatic tachycardia syndrome, low blood pressure, low glucose level, deep vein thrombosis (DVT), stroke, pulmonary embolism (PE), superficial thrombophlebitis, blood clotting, tension pneumothorax, supraventricular tachycardia (SVT), idiopathic intermittent atrial fibrillation, angina, myocardial infarction (MI), hyperglycemia, diabetic ketoacidosis (DKT), and other disorders.

Following analyzing a physiological structure's vital signs and internal parts, and diagnosing the physiological structure for any disorders, the measuring device may use all of the data of the physiological structure to predict behaviors of the physiological structure. In one example, a person who is climbing a mountain, i.e. mountain climber, may have a measuring device attached to their waist to monitor their vital signs. After the measuring device has obtained and analyzed data of the mountain climber's vital signs, the measuring device may determine that the mountain climber is at risk of experiencing an illness, injury, disorder, etc. The measuring device may provide an alert and message to the mountain climber to notify the mountain climber of the risks and provide recommendations of how to prevent such illness, injury and/or disorder. Such recommendations may include instructing the climber to stop moving and rest, decrease elevation, contact the base camp medical provider, or take medicine.

In another example, a measuring device attached to a pilot's arm may send an alert to the pilot when the measuring device determines, based on data of the pilot's vital signs, that the pilot is in danger of losing consciousness if the pilot continues to perform dangerous flying maneuvers with the plane.

In another example, a measuring device may be attached to a pregnant woman who is in her second or third trimester. The measuring device may be used to monitor the pregnant woman's vital signs to detect longitudinal change in blood pressure. When increases in blood pressure are detected, the measuring device may send an alert and/or message to a doctor so further analysis may be conducted. Early detection of changes in blood pressure in pregnant women may allow for the prevention and quick diagnosis of eclampsia and preeclampsia, thus allowing for medical attention and treatment to be applied more quickly.

In another example, a measuring device attached to an elderly man to monitor the elderly man's vital signs. The measuring device may determine, based on data of the elderly man's vital signs, that the elderly man will fall (e.g., rapidly progressive hypotension and/or tachycardia plus or minus accelerometry). The measuring device may send an alert and/or message to the elderly man to notify the elderly man that he is about to fall so that he may sit or lie down prior to collapsing. The measuring device may also include an accelerometer to detect when the elderly man has fallen.

The measuring device may also send an alert and/or message to other individuals, such as family members, doctors, emergency responders, etc., who have been authorized and listed in the measuring device to send alerts and messages to. The measuring device may be able to predict a variety of illnesses, disorders, injuries, etc., that a physiological structure may experience based on data of the physiological structure's vital signs and internal parts. The measuring device may also use all of the data of the physiological structure that it has obtained to determine that particular illnesses, disorders, injuries, conditions, etc., are present in the physiological structure. In this way, the measuring device may both predict future and determine present attributes and conditions relating to the vital signs, internal parts, and overall health of a physiological structure.

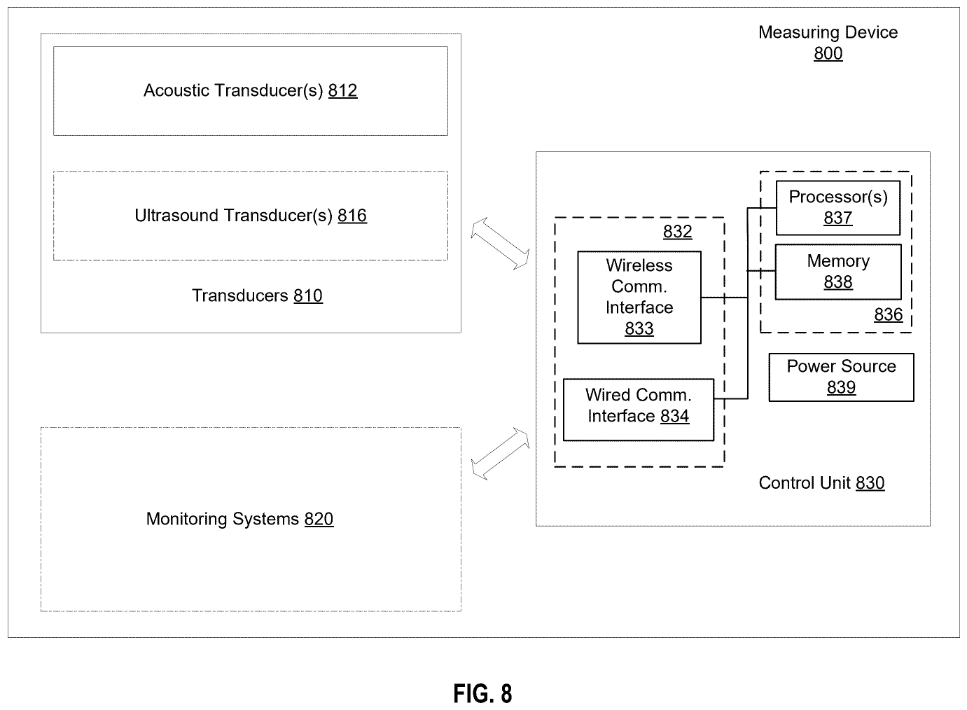

FIG. 8 illustrates an example measuring device 800, in accordance with various embodiments of the presently disclosed technology.

As depicted, measuring device 800 comprises a control unit 830 and transducers 810. In some embodiments, measuring device 800 may further comprise a monitoring system 820 (described in greater detail below).

As depicted, transducers 810 comprise acoustic transducer(s) 812. Acoustic transducer(s) 812 may comprise one or more acoustic transducers. As used herein, an acoustic transducer may refer to a device that: (a) transmits acoustic energy (e.g., a speaker): (b) acquires/receives acoustic energy (e.g., a microphone): or (c) transmits and receives acoustic energy (e.g., a transceiver than includes both acoustic transmitter and acoustic receiver components). An acoustic transducer that transmits acoustic energy may convert received electrical signals into the acoustic energy/acoustic signals it transmits. An acoustic transducer that acquires/receives acoustic energy may convert acquired/received acoustic energy into electrical signals. As used herein, an acoustic transducer that transmits acoustic energy but does not receive/acquire acoustic energy (e.g., a speaker) may be referred to as a non-receiver acoustic transducer. In general, non-receiver acoustic transducers (e.g., speakers) are less expensive and consume less power than acoustic transducers that both transmit and receive acoustic energy (e.g., acoustic transceivers).

Certain embodiments can reduce expensive and power consumption by using non-receiver acoustic transducers in measuring devices of the presently disclosed technology. For example, acoustic transducer(s) 812 may comprise one or more non-receiver acoustic transducers. Such a non-receiver acoustic transducer can be used to transmit acoustic energy of different frequencies towards a blood vessel in order to probe/determine the blood vessel's resonant frequency. In general, the blood vessel will absorb some of the acoustic energy, and reflect some of the acoustic energy back. As embodiments of the presently disclosed technology are designed in appreciation of, when the blood vessel is hit with acoustic energy at a resonant frequency of a wall of the blood vessel (i.e., a blood vessel wall), a significantly greater amount of the acoustic energy will be absorbed by the blood vessel wall, and less acoustic energy will be reflected back at the non-receiver acoustic transducer. For example, embodiments can use the non-receiver acoustic transducer to transmit acoustic energy at a first frequency and then transmit acoustic energy at a second frequency. Here, the second frequency may correspond with the resonant frequency of the blood vessel wall. Accordingly, responsive to transmission of acoustic energy at the second frequency, the blood vessel wall may reflect significantly less acoustic energy back to the non-receiver acoustic transducer.

However, lacking an acoustic energy receiving component, the non-receiver acoustic transducer would generally not be able to detect the change in acoustic energy that is reflected back to the non-receiver acoustic transducer when the blood vessel is hit with acoustic energy at its resonant frequency (i.e., the second frequency). Embodiments are intelligently designed to overcome this technical challenge by measuring an electrical property of the non-receiver acoustic transducer (e.g., current, voltage, power, resistance, impedance, etc.) as a proxy for measuring the acoustic energy that is reflected back to the non-receiver acoustic transducer. This is based on an intelligent insight that electrical properties of the non-receiver acoustic transducer may be impacted as a function of the magnitude of acoustic energy propagating towards the non-receiver acoustic transducer in a direction that opposes the direction the non-receiver acoustic transducer transmits audio energy (akin to pushing a door into the wind). For example, the amount of electrical power the non-receiver acoustic transducer requires to transmit acoustic energy may increase when other acoustic energy is propagating back towards the non-receiver acoustic transducer in an opposing direction. In other words, the non-receiver acoustic transducer may require less electrical power to transmit acoustic energy when a lesser magnitude of acoustic energy is reflected back to the non-receiver acoustic transducer from the blood vessel wall. Accordingly, embodiments can determine a resonant frequency of the blood vessel wall as the transmission frequency at which an amount of power required to transmit is reduced/minimized.

However, it should be understood that in other embodiments acoustic transducer(s) 812 may comprise acoustic energy transceivers that can both transmit and receive acoustic energy. In these embodiments, measuring device 800 can determine the resonant frequency of the blood vessel wall by analyzing acoustic energy that is received by acoustic transducer(s) 812 after being reflected back from the blood vessel.

As depicted, in certain embodiments transducers 810 may also comprise ultrasound transducer(s) 816. Ultrasound transducer(s) 816 may comprise one or more ultrasound transducers capable of transmitting and receiving ultrasound energy (i.e., acoustic energy involving ultrasound signals). While ultrasound transducer(s) 816 may be more expensive and consume more power than non-ultrasound acoustic transducers, ultrasound transducer(s) 816 can be useful for imagining the blood vessel. Accordingly, measuring device 800 can utilize the imagining capabilities of ultrasound energy to determine wall thickness of the blood vessel and blood vessel radius of the blood vessel. As alluded to above, measuring device 800 can use these determined parameters, along with determined resonant frequency of the blood vessel wall, to generate a blood pressure measurement. While wall thickness and blood vessel radius generally remain relatively consistent across individuals, precise/individualized measurements of these parameters can lead to more accurate blood pressure measurements. Accordingly, leveraging ultrasound transducer(s) 816 to determine wall thickness of the blood vessel and blood vessel radius of the blood vessel, measuring device 800 can improve blood pressure measurement accuracy. However, in other embodiments (e.g., where ultrasound transducer(s) 816 are not included in transducers 810 (e.g., to reduce cost and power consumption), measuring device 800 can rely on estimated values for these parameters acquired from applicable medical databases/medical literature. In still further embodiments, measuring device 800 can utilize ultrasound transducer(s) 816 to initially measure wall thickness and blood vessel radius of the blood vessel, and then rely on lower power-consuming non-ultrasound transducers for continued monitoring of blood vessel wall resonant frequency as embodiments do not require ultrasound transducers for such monitoring.

As depicted, measuring device 800 also comprises a control unit 830. Components within control unit 830 can communicate via a data bus, and/or other suitable communication interfaces.

Communication circuit 832 may comprise at least one of a wireless communication interface 833 (e.g., a transceiver with an antenna) and a wired communication interface 834 (e.g., an I/O interface with an associated hardwired data port). Control unit 830 can utilize communication circuit 832 to communicate with transducers 810 and monitoring systems 820. Control unit 830) can also utilize communication circuit 832 to communicate with devices remote from measuring device 800. For example, in certain implementations monitoring systems 820 may be located remotely from measuring device 800.

Wireless communication interface 833 may include a transceiver (i.e., a receiver and transmitter) to allow wireless communications via various communication protocols such as, WiFi, Zigbee, Bluetooth, near field communications, etc. As alluded to above, wireless communication interface 833 may comprise an antenna coupled to the transceiver to send and receive radio signals wirelessly. These radio signals can include information sent to and from transducers 810 and monitoring systems 820. These radio signals can also include radio signals sent to and from devices remote from measuring device 800.

Wired communication interface 834 can include a receiver and a transmitter for hardwired communications with other components of measuring device 800 (e.g., transducers 810 and monitoring systems 820). For example, wired communication interface 834 can provide a hardwired interface to other components, including transducers 810 and monitoring systems 820. Wired communication interface 834 can communicate with these components using Ethernet or any number of other wired communication protocols. In various examples, wired communication interface 834 can communicate with devices remote from measuring device 800.

As depicted, determination circuit 836 includes processor(s) 837 and memory 838. Processor(s) 837 can include one or more processing resources, such as GPUs, CPUs, microprocessors, etc.

Memory 838 may comprise one or more modules of various forms of memory/data storage (e.g., flash, RAM, etc.) for storing the various data, parameters, and operational instructions utilized by processor(s) 837, as well as any other suitable information.

While the specific example of FIG. 8 is illustrated using processor and memory circuitry, determination circuit 836 can be implemented utilizing any form of circuitry including, for example, hardware, software, or a combination thereof. As a further example, one or more processors, controllers, ASICs, PLAS, PALs, CPLDs, FPGAs, logical components, software routines or other mechanisms may be used to implement control unit 830.

Power source 839 may comprise any type of suitable power source. For instance, power supply 839 can include one or more batteries (e.g., rechargeable or primary batteries comprising Li-ion, Li-Polymer, NiMH, NiCd, NiZn, NiH2, etc.), a power connector (e.g., to connect to supplied power), and an energy harvester (e.g., solar cells, piezoelectric system, etc.).

As alluded to above, in certain embodiments measuring device 800 may include monitoring systems 820. In other embodiments, monitoring systems 820 may comprise a remotely located, separate system. Monitoring systems 820 can display information to users related to monitored blood pressure. For example, control unit 830 can generate a blood pressure measurement, and then send a notification to monitoring systems 820 that contains the generated blood pressure measurement. Monitoring systems 820 may comprise a graphical user interface (GUI) that displays the notification to a user.

In some implementations, measuring device 800 may comprise a wearable cuff dimensioned to be worn around a person's limb (e.g., an armband). The blood vessel may be located within the person's limb. Here, transducers 810 may be mechanically attached to the wearable cuff such that they contact the person's tissue.

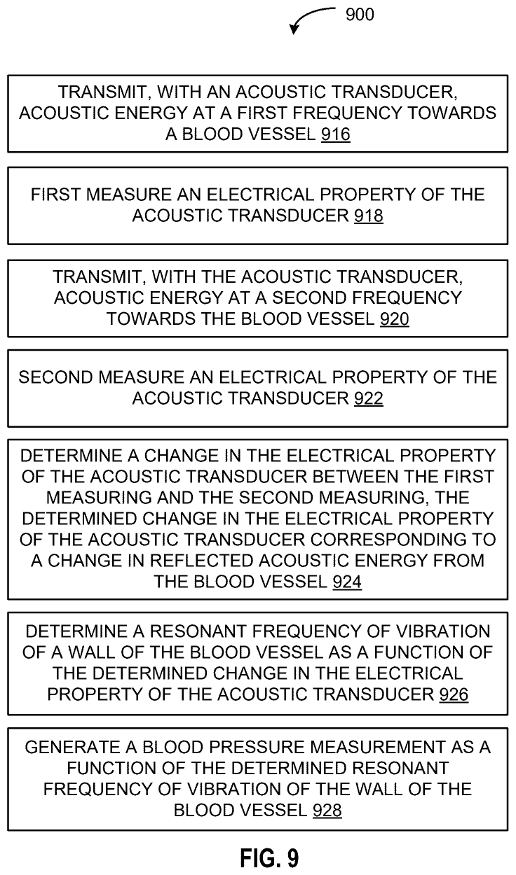

FIG. 9 illustrates an example methodology 900 for generating a blood pressure measurement as a function of a determined resonant frequency of vibration of a wall of a blood vessel.

As depicted, operation 916 may comprise transmitting, with an acoustic transducer, acoustic energy at a first frequency towards a blood vessel (e.g., an artery, a vein, a capillary, etc.). In certain embodiments, the acoustic transducer may comprise a non-receiver acoustic transducer. In various implementations, the blood vessel may be in a physiological structure and transmitting acoustic energy towards the blood vessel can comprise transmitting acoustic energy towards the blood vessel through the physiological structure.

Operation 918 may comprise first measuring an electrical property of the acoustic transducer. As alluded to above, the electrical property may comprise at least one of: current; power: voltage: resistance; and impedance. Here, first measuring the electrical property of the acoustic transducer may be responsive to transmission of the acoustic energy at the first frequency.

Operation 920 may comprise transmitting, with the acoustic transducer, acoustic energy at a second frequency towards the blood vessel. In certain examples, the second frequency may correspond with the resonant frequency of vibration of the wall of the blood vessel.

Operation 922 may comprise second measuring the electrical property of the acoustic transducer. Second measuring the electrical property of the acoustic transducer may be responsive to transmission of the acoustic energy at the second frequency.

Operation 924 may comprise determining a change in the electrical property of the acoustic transducer between the first measuring and the second measuring, the determined change in the electrical property of the acoustic transducer corresponding to a change in reflected acoustic energy from the blood vessel.

Operation 926 may comprise determining a resonant frequency of vibration of a wall of the blood vessel as a function of the determined change in the electrical property of the acoustic transducer.

Operation 928 may comprise generating a blood pressure measurement as a function of the determined resonant frequency of vibration of the wall of the blood vessel. In certain implementations, generating the blood pressure measurement may comprise generating the blood pressure measurement as a function of: the determined resonant frequency of vibration of the wall of the blood vessel: estimated wall thickness of the blood vessel; and estimated vessel radius of the blood vessel.

In certain implementations, methodology 900 may further comprise sending, to a monitoring system, a notification containing the generated blood pressure measurement.

In some implementations, methodology 900 may further comprise: (a) transmitting, with the acoustic transducer, acoustic energy at a third frequency towards the blood vessel: (b) third measuring the electrical property of the acoustic transducer; and (c) determining a second change in the electrical property of the acoustic transducer between the second measuring and the third measuring, the determined second change in the electrical property of the transducer corresponding to a second change in reflected acoustic energy from the blood vessel. Here, determining the resonant frequency of vibration of the wall of the blood vessel may comprise determining the resonant frequency of vibration of the wall of the blood vessel as a function of the first and second determined changes in reflected acoustic energy from the blood vessel. Use of the third frequency here may result in improved accuracy/precision for determining the resonant frequency of vibration of the wall of the blood vessel.

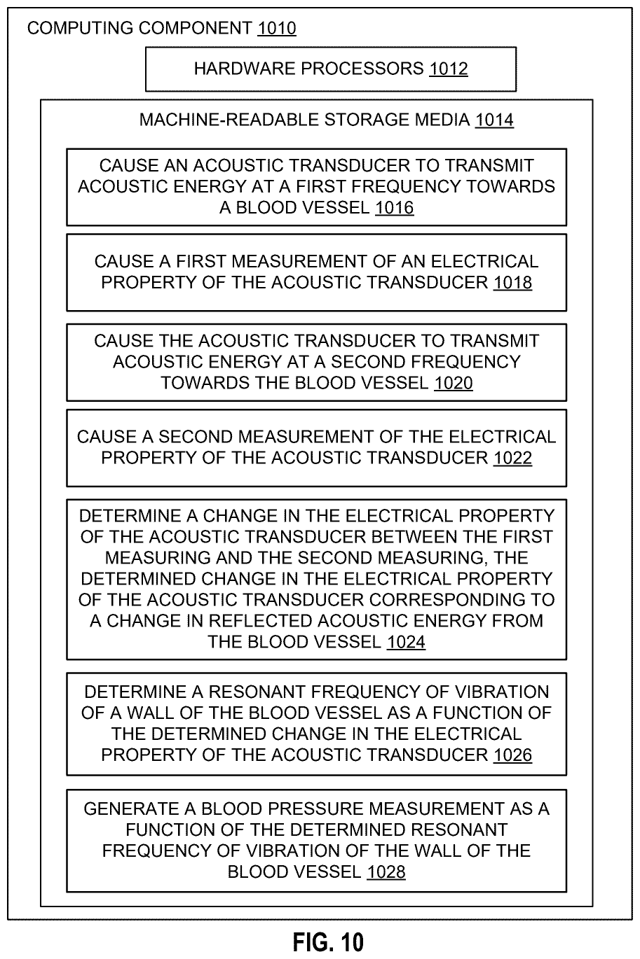

FIG. 10 is a companion figure to FIG. 9 that depicts how the methodology 900 may be implemented using a computing component 1010. Here, computing component 1010 may be implemented on control unit 830 of FIG. 8. While the instructions of FIG. 10 will not be described again for brevity, other components of computing component 1010 are described here.

Computing component 1010 may be, for example, a server computer, a controller, or any other similar computing component capable of processing data. In the example implementation of FIG. 10, the computing component 1010 includes a hardware processor 1012, and machine-readable storage medium for 1014.

Hardware processor 1012 may be one or more central processing units (CPUs), semiconductor-based microprocessors, and/or other hardware devices suitable for retrieval and execution of instructions stored in machine-readable storage medium 1014. Hardware processor 1012 may fetch, decode, and execute instructions, such as instructions 1016-1028, to control processes or operations. As an alternative or in addition to retrieving and executing instructions, hardware processor 1012 may include one or more electronic circuits that include electronic components for performing the functionality of one or more instructions, such as a field programmable gate array (FPGA), application specific integrated circuit (ASIC), or other electronic circuits.

A machine-readable storage medium, such as machine-readable storage medium 1014, may be any electronic, magnetic, optical, or other physical storage device that contains or stores executable instructions. Thus, machine-readable storage medium 1014 may be, for example, Random Access Memory (RAM), non-volatile RAM (NVRAM), an Electrically Erasable Programmable Read-Only Memory (EEPROM), a storage device, an optical disc, and the like. In some examples, machine-readable storage medium 1014 may be a non-transitory storage medium, where the term “non-transitory” does not encompass transitory propagating indicators. As described in detail below, machine-readable storage medium 1014 may be encoded with executable instructions, for example, instructions 1016-1028.

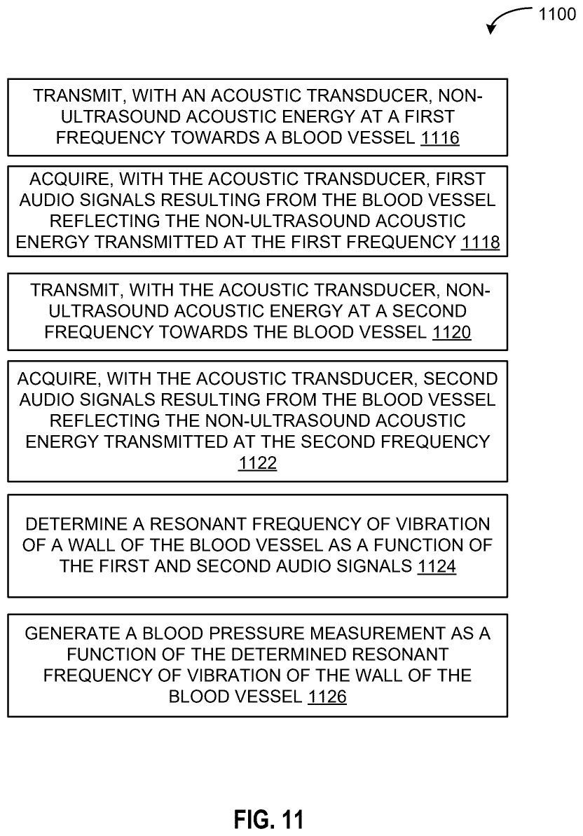

FIG. 11 illustrates an example methodology 1100 for generating a blood pressure measurement as a function of a determined resonant frequency of vibration of a wall of a blood vessel.

Operation 1116 may comprise transmitting, with an acoustic transducer, non-ultrasound acoustic energy at a first frequency towards a blood vessel. As alluded to above, generating non-ultrasound acoustic energy instead of ultrasound acoustic energy can reduce expense and power consumption.

Operation 1118 may comprise acquiring, with the acoustic transducer, first audio signals resulting from the blood vessel reflecting the non-ultrasound acoustic energy transmitted at the first frequency.

Operation 1120 may comprise transmitting, with the acoustic transducer, non-ultrasound acoustic energy at a second frequency towards the blood vessel.

Operation 1122 may comprise acquiring, with the acoustic transducer, second audio signals resulting from the blood vessel reflecting the non-ultrasound acoustic energy transmitted at the second frequency.

Operation 1124 may comprise determining a resonant frequency of vibration of a wall of the blood vessel as a function of the first and second audio signals.

Operation 1126 may comprise generating a blood pressure measurement as a function of the determined resonant frequency of vibration of the wall of the blood vessel. Here, generating the blood pressure measurement may comprise generating the blood pressure measurement as a function of: the determined resonant frequency of vibration of the wall of the blood vessel: estimated wall thickness of the blood vessel; and estimated vessel radius of the blood vessel. As alluded to above, wall thickness and vessel radius may be estimated here to account for lack of imagining by ultrasound for determining these parameters.

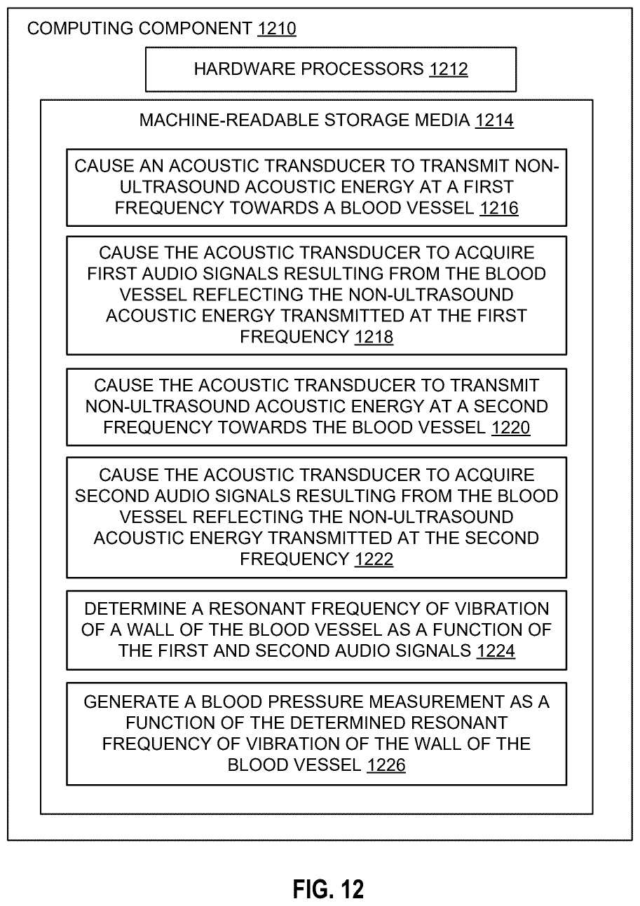

FIG. 12 is a companion figure to FIG. 11 that illustrates how methodology 1100 may be performed by a computing component 1210. Like computing component 1010, computing component 1210 may be implemented on control unit 830 of FIG. 8. The instructions of FIG. 12 (i.e., instructions 1214-1226) will not be described again for brevity. Hardware processor 1212 and machine-readable storage media 1214 may be the same/similar as hardware processor 1012 and machine-readable storage media 1014 of FIG. 10.

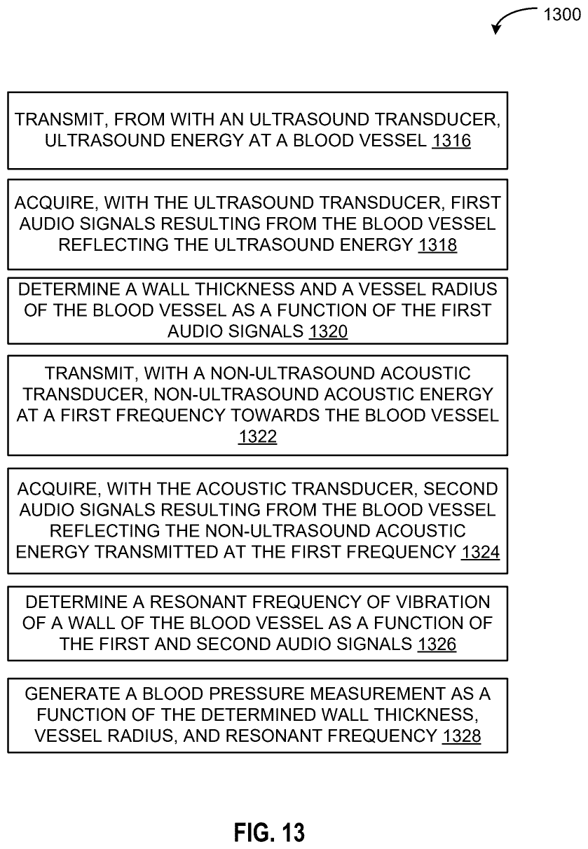

FIG. 13 illustrates an example methodology 1300 for generating a blood pressure measurement as a function of a determined resonant frequency of vibration of a wall of a blood vessel.

Operation 1316 may comprise transmitting, from with an ultrasound transducer, ultrasound energy at a blood vessel.

Operation 1318 may comprise acquiring, with the ultrasound transducer, first audio signals resulting from the blood vessel reflecting the ultrasound energy.

Operation 1320 may comprise determining a wall thickness and a vessel radius of the blood vessel as a function of the first audio signals.

Operation 1322 may comprise transmitting, with a non-ultrasound acoustic transducer, non-ultrasound acoustic energy at a first frequency towards the blood vessel.

Operation 1324 may comprise acquiring, with the acoustic transducer, second audio signals resulting from the blood vessel reflecting the non-ultrasound acoustic energy transmitted at the first frequency.

Operation 1326 may comprise determining a resonant frequency of vibration of a wall of the blood vessel as a function of the first and second audio signals.

Operation 1328 may comprise generating a blood pressure measurement as a function of the determined wall thickness, vessel radius, and resonant frequency.

As alluded to above, embodiments can conserve power consumption by only using an ultrasound transducer to initially determine blood vessel wall thickness and blood vessel radius (which generally cannot be determined using non-ultrasound transducers), and then only using non-ultrasound transducers for continued monitoring/determination of resonant frequency of vibration of the wall of the blood vessel.

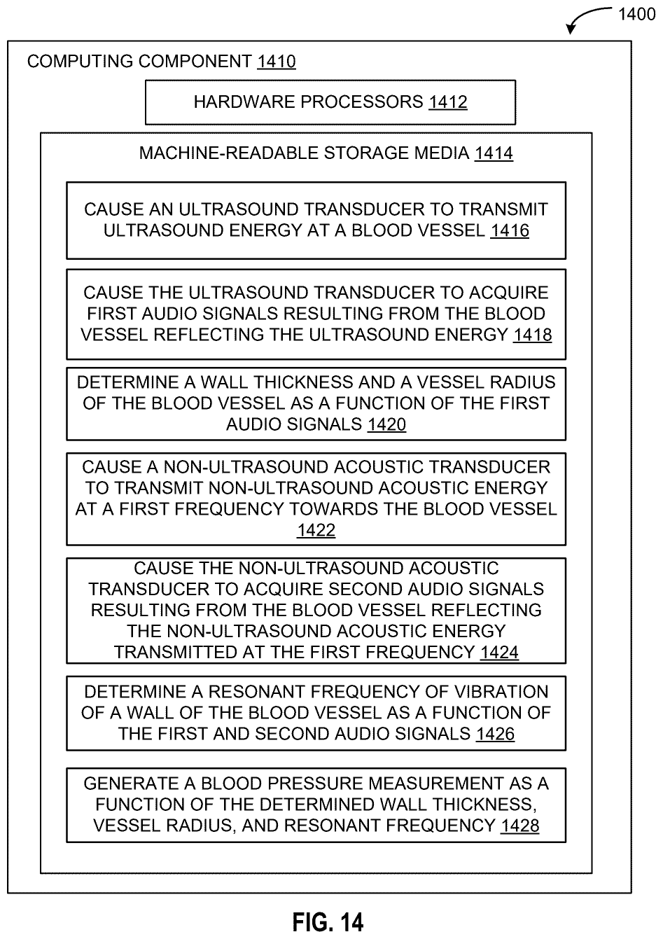

FIG. 14 is a companion figure to FIG. 13 that illustrates how methodology 1300 may be performed by a computing component 1410. Like computing component 1010, computing component 1410 may be implemented on control unit 830 of FIG. 8. The instructions of FIG. 14 (i.e., instructions 1414-1428) will not be described again for brevity. Hardware processor 1412 and machine-readable storage media 1414 may be the same/similar as hardware processor 1012 and machine-readable storage media 1014 of FIG. 10.

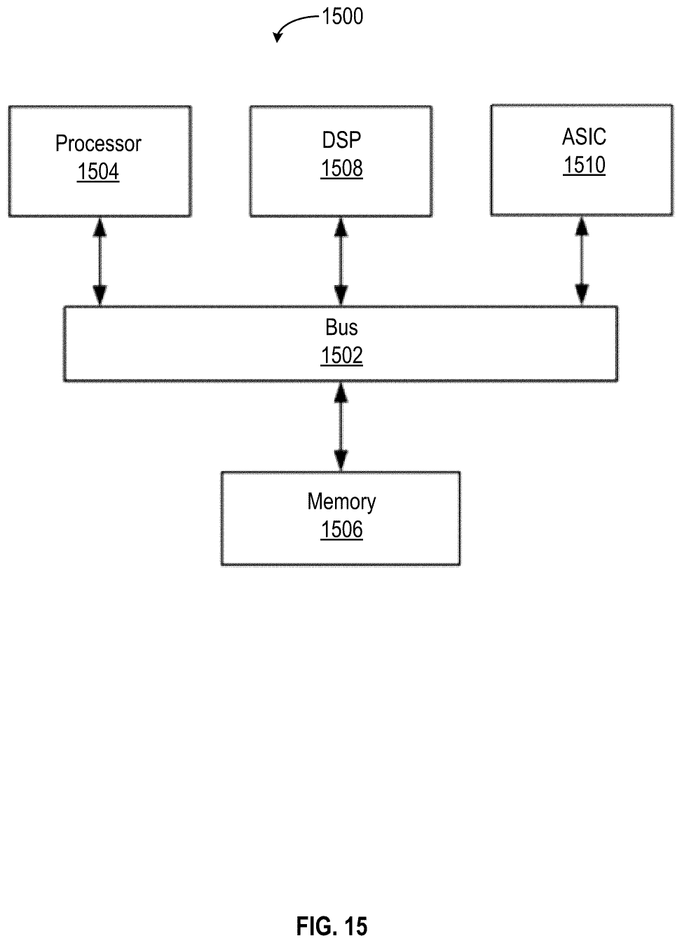

FIG. 15 illustrates a chip set 1500 in which embodiments of the disclosure may be implemented. Chip set 1500 can include, for instance, processor and memory components incorporated in one or more physical packages. By way of example, a physical package includes an arrangement of one or more materials, components, and/or wires on a structural assembly (e.g., a baseboard) to provide one or more characteristics such as physical strength, conservation of size, and/or limitation of electrical interaction.

In one embodiment, chip set 1500 includes a communication mechanism such as a bus 1502 for passing information among the components of the chip set 1500. A processor 1504 has connectivity to bus 1502 to execute instructions and process information stored in a memory 1506. Processor 1504 includes one or more processing cores with each core configured to perform independently. A multi-core processor enables multiprocessing within a single physical package. Examples of a multi-core processor include two, four, eight, or greater numbers of processing cores. Alternatively or in addition, processor 1504 includes one or more microprocessors configured in tandem via bus 1502 to enable independent execution of instructions, pipelining, and multithreading. Processor 1504 may also be accompanied with one or more specialized components to perform certain processing functions and tasks such as one or more digital signal processors (DSP) 1508, and/or one or more application-specific integrated circuits (ASIC) 1510. DSP 1508 can typically be configured to process real-world signals (e.g., sound) in real time independently of processor 1504. Similarly, ASIC 1510 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

Processor 1504 and accompanying components have connectivity to the memory 1506 via bus 1502. Memory 1506 includes both dynamic memory (e.g., RAM) and static memory (e.g., ROM) for storing executable instructions that, when executed by processor 1504, DSP 1508, and/or ASIC 1510, perform the process of example embodiments as described herein. Memory 1506 also stores the data associated with or generated by the execution of the process.

In this document, the terms “machine readable medium,” “computer readable medium,” and similar terms are used to generally refer to non-transitory mediums, volatile or non-volatile, that store data and/or instructions that cause a machine to operate in a specific fashion. Common forms of machine readable media include, for example, a hard disk, solid state drive, magnetic tape, or any other magnetic data storage medium, an optical disc or any other optical data storage medium, any physical medium with patterns of holes, a RAM, a PROM, EPROM, a FLASH-EPROM, NVRAM, any other memory chip or cartridge, and networked versions of the same.

These and other various forms of computer readable media may be involved in carrying one or more sequences of one or more instructions to a processing device for execution. Such instructions embodied on the medium, are generally referred to as “instructions” or “code.” Instructions may be grouped in the form of computer programs or other groupings. When executed, such instructions may enable a processing device to perform features or functions of the present application as discussed herein.

In this document, a “processing device” may be implemented as a single processor that performs processing operations or a combination of specialized and/or general-purpose processors that perform processing operations. A processing device may include a CPU, GPU, APU, DSP. FPGA, ASIC, SOC, and/or other processing circuitry. The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A device for continuous blood pressure monitoring, comprising:
a wearable acoustic unit configured to non-invasively detect an acoustic signal from a blood vessel of a subject; and
a processing unit configured to use said acoustic signal to continuously monitor a blood pressure of the subject at an error rate of less than or equal to about two percent.

2. The device of claim 1, wherein the device comprises an armband, wristband, leg band, or adhesive patch, and wherein said wearable acoustic unit is coupled to said armband, wristband, or adhesive patch.

3. The device of claim 1, wherein said wearable acoustic unit comprises an acoustic transducer.

4. The device of claim 3, wherein said acoustic transducer is an acoustic transceiver.

5. The device of claim 1, wherein said wearable acoustic unit comprises a plurality of acoustic transducers.

6. The device of claim 1, wherein said wearable acoustic unit comprises an ultrasound transducer.

7. The device of claim 1, wherein said wearable acoustic unit is configured to direct acoustic energy to said blood vessel to generate said acoustic signal.

8. The device of claim 1, wherein said wearable acoustic unit is configured to image said blood vessel.

9. The device of claim 1, wherein said processing unit is further configured to monitor average blood pressure of said subject.

10. The device of claim 9, wherein said device is configured to detect changes in said average blood pressure of less than 10 millimeters of mercury (mmHg).

11. A method for continuous blood pressure monitoring, comprising:
(a) using a wearable acoustic unit to non-invasively detect an acoustic signal from a blood vessel of a subject; and
(b) processing said acoustic signal to continuously monitor a blood pressure of said subject at an error rate of less than or equal to about two percent.

12. The method of claim 11, wherein said wearable acoustic unit comprises an acoustic transducer.

13. The method of claim 12, further comprising using said acoustic transducer to direct acoustic energy to said blood vessel, wherein interaction of said acoustic energy with said blood vessel generates said acoustic signal.

14. The method of claim 11, wherein said wearable acoustic unit comprises an ultrasound transducer.

15. The method of claim 14, further comprising using said ultrasound transducer to determine a radius of said blood vessel and a wall thickness of said blood vessel.

16. The method of claim 15, further comprising using Laplace's method to determine said radius and said wall thickness.

17. The method of claim 15, further comprising using said blood vessel and said wall thickness to continuously monitor said blood pressure.

18. The method of claim 11, further comprising using said wearable acoustic unit to image said blood vessel.

19. The method of claim 11, further comprising monitoring an average blood pressure of said subject.

20. The method of claim 19, wherein said method is usable to detect changes in said average blood pressure of less than 10 mmHg.

21. The method of claim 19, further comprising using a low-pass filter to remove high frequency blood pressure artifacts from said average blood pressure to increase accuracy of said average blood pressure as compared to an average blood pressure monitored without said high frequency blood pressure artifacts removed.

22. The method of claim 11, further comprising monitoring said blood pressure of said subject over a time period to determine longitudinal trends in blood pressure of said subject over said period of time.

23. The method of claim 22, further comprising comparing said longitudinal trends in blood pressure of said subject with other longitudinal trends in blood pressure of other subjects to generate a comparison of longitudinal trends.

24. The method of claim 23, further comprising using said comparison of longitudinal trends to determine a health status, predicted disease progression, or predicted treatment outcome for said subject.

25. The method of claim 23, wherein said comparison of longitudinal trends is generated using machine learning.

26. The method of claim 24, further comprising using machine learning to determine said health status, said predicted disease progression, or said predicted treatment outcome for said subject from said comparison of longitudinal trends.

27. The method of claim 26, further comprising modifying a treatment regimen of said subject based at least in part on said health status, said predicted disease progression, or said predicted treatment outcome for said subject.
